SEQUENCE LISTING (23 sequences)

SEQ ID NO: 1 - protein, 6 aa, Artificial Sequence
  Synthesized Polypeptide
XXXXXX

SEQ ID NO: 2 - protein, 6 aa, Artificial Sequence
  Synthesized Polypeptide
FFVIFY

SEQ ID NO: 3 - protein, 4 aa, Artificial Sequence
  Synthesized Polypeptide
RRKK

SEQ ID NO: 4 - protein, 10 aa, Artificial Sequence
  Synthesized Polypeptide
RRKKFFVIFY

SEQ ID NO: 5 - protein, 6 aa, Artificial Sequence
  Synthesized Polypeptide
WLVFFV

SEQ ID NO: 6 - protein, 10 aa, Artificial Sequence
  Synthesized Polypeptide
WLVFFVRRKK

SEQ ID NO: 7 - protein, 10 aa, Artificial Sequence
  Synthesized Polypeptide
FFVIFYRRKK

SEQ ID NO: 8 - protein, 6 aa, Artificial Sequence
  Synthesized Polypeptide
IFYFFR

SEQ ID NO: 9 - protein, 6 aa, Artificial Sequence
  Synthesized Polypeptide
WLVFFV

SEQ ID NO: 10 - protein, 6 aa, Artificial Sequence
  Synthesized Polypeptide
IAYFAR

SEQ ID NO: 11 - protein, 12 aa, Artificial Sequence
  Synthesized Polypeptide
WLVFFVIAYFAR

SEQ ID NO: 12 - protein, 16 aa, Artificial Sequence
  Synthesized Polypeptide
WLVFFVIFYFFRRRKK

SEQ ID NO: 13 - protein, 15 aa, Artificial Sequence
  Synthesized Polypeptide
RRKKWLVFFVIYFFR

SEQ ID NO: 14 - protein, 12 aa, Artificial Sequence
  Synthesized Polypeptide
IVWFYLFRFFVF

SEQ ID NO: 15 - protein, 10 aa, Artificial Sequence
  Synthesized Polypeptide
FFVIAYRRKK

SEQ ID NO: 16 - protein, 9 aa, Artificial Sequence
  Synthesized Polypeptide
FFVIAYFAR

SEQ ID NO: 17 - protein, 13 aa, Artificial Sequence
  Synthesized Polypeptide
FFVIAYFARRRKK

SEQ ID NO: 18 - protein, 12 aa, Artificial Sequence
  Synthesized Polypeptide
WLVFFVIFYFFR

SEQ ID NO: 19 - protein, 10 aa, Artificial Sequence
  Synthesized Polypeptide
IFYFFRRRKK

SEQ ID NO: 20 - protein, 12 aa, Artificial Sequence
  Synthesized Polypeptide
WLVFFVIFYFFR

SEQ ID NO: 21 - protein, 16 aa, Artificial Sequence
  Synthesized Polypeptide
RRKKWLVFFVIFYFFR

SEQ ID NO: 22 - protein, 6 aa, Artificial Sequence
  Synthesized Polypeptide
FFVIAY

SEQ ID NO: 23 - protein, 6 aa, Artificial Sequence
  Synthesized Polypeptide
IAYFAR

(19) United States
(12) Patent Application Publication
(10) Pub. No.: US 2013/0205416 A1
(43) Pub. Date: Aug. 8, 2013

(54) ANTI-VIRAL AGENT

(71) Applicants: Anthony Nash, Edinburgh (GB); Bernadette Dutia, Edinburgh (GB); Mark Stevens, Peebles (GB)

(72) Inventors: Anthony Nash, Edinburgh (GB); Bernadette Dutia, Edinburgh (GB); Mark Stevens, Peebles (GB)

(21) Appl. No.: 13/812,252

(22) PCT Filed: Jul. 28, 2011
(86) PCT No.: PCT/GB2011/051436

§ 371 Date: Apr. 5, 2013

(30) Foreign Application Priority Data
Jul. 28, 2010 (GB) 1012651.4

(51) Int. Cl. C07K 7/08; C07K 5/11; G01N 33/50; C07K 7/06

(52) U.S. Cl. CPC C07K 7/08; C07K 7/06; C07K 5/1019; G01N 33/5008
USPC 800/13; 514/3.7; 536/23.1; 514/44 R; 424/93.2; 530/329; 530/330; 530/328; 530/327; 530/326; 435/320.1; 435/5; 435/252.3; 800/8

(57) ABSTRACT

The present invention provides an agent, in particular a peptide, of formula A, comprising an amino acid sequence X1-X2-X3-X4-X5-X6 (SEQ ID NO 1) wherein X1 can be phenylalanine, isoleucine or tryptophan; X2 can be leucine or phenylalanine or alanine; X3 can be tyrosine or valine; X4 can be leucine, phenylalanine or isoleucine; 10 X5 can be phenyalanine or alanine; and X6 can be valine, arginine or tyrosine, or a fragment or variant of the peptide, wherein said peptide fragment or variant is capable of specifically binding to haemagglutinin, to inhibit the binding of a virus having haemagglutinin on its surface, for use in the treatment of a virus, for example influenza.

### FIELD OF THE INVENTION

The present invention relates to an agent, more specifically to a peptide or a peptide mimetic useful for the treatment of viruses that have haemagglutinin on their coat surface, including haemagglutinin-neuraminidase structures on their surface, in particular a peptide or mimetic thereof useful for the treatment of influenza. Further, the present invention relates to a composition comprising said agent, in particular a peptide or peptide mimetic and the use of the agent or composition as a prophylactic treatment prior to and/or treatment following infection by a virus that has haemagglutinin on its surface, for example influenza.

### BACKGROUND OF THE INVENTION

Several viruses are known to include haemagglutinin on their surface and this is believed to enable attachment of such virus to sialic acid-containing cell receptors and thereby initiate infection.

Influenza, commonly referred to as the flu, is an infectious disease caused by RNA viruses. Haemagglutinin (HA) and Neuraminidase (NA) are two large glycoproteins on the outside of the influenza virus particles. HA is considered to mediate binding of the virus to sialic acid sugars on target cells, while NA is considered to mediate release of virus from infected cells.

In virus classification, influenza viruses have been designated influenza virus A, influenza virus B and influenza virus C. Influenza virus A can be subdivided into different serotypes based on antibody response, for example H1N1, H2N2, H3N2, H5N1, H7N7, H1N2, H9N2, H7N2, H7N3 and H10N7. Influenza B almost exclusively infects humans, whilst influenza C infects humans, dogs and pigs, but is less common than influenza A or B.

In the West, seasonal influenza is responsible for a number of deaths, for example in the USA around 30,000 people die annually from influenza infection with up to 200,000 hospitalised. In the case of a pandemic, these figures would be significantly larger and if the prevalent influenza strain was particularly virulent it has been estimated by the British Medical Journal that such an outbreak would cost the UK around £72 billion.

At present, several antiviral drugs have been developed that are active in controlling influenza and blocking transmission. Broadly, these can be divided into the following categories:

1. Neuraminidase inhibitors (NAI's, such as oseltamivir (Tamiflu™) and zanamivir (Relenza™)). These block the exit of virus from the infected cell.
2. Adamantane derivatives (e.g. amantadine (Symmetrel™) and rimantadine (Flumadine™)). These block the function of the influenza virus M2 ion channel protein.
3. Agents that block viral entry into a cell.
4. Other agents that block influenza infection in the cell e.g. inhibitors of RNA polymerase and siRNA.

### SUMMARY OF THE INVENTION

The present invention relates to an agent of formula A for use in the treatment of viruses which include haemagglutinin on their surface, for example influenza virus, measles virus, mumps virus, parainfluenza virus, respiratory syncytial virus (RSV), rubella virus, rabies virus, nipah virus, hendra virus, canine distemper virus, phocine distemper virus, rinderpest virus, Newcastle disease virus, Sendai virus or metapneumovirus.

Suitably, the present invention may relate to an agent of formula A for use in the treatment of viruses selected from respiratory syncytial virus, metapneumovirus and influenza, in particular metapneumovirus and influenza.

An agent of the present invention may be useful in binding to phytohaemagglutinin.

In particular, the present invention relates to an agent of formula A for use in the treatment of influenza.

An agent of formula A may be capable of specifically binding to a specific haemagglutinin of a virus, in particular to haemagglutinin of influenza, more particularly to haemagglutinin of influenza type A, more particularly influenza types H1N1, H3N2, H5N1 and H7N1, more particularly influenza A/WSN/33 H1N1, A/PR8/34 H1N1, A/England/195/09/PR8H1N1, A/Victoria/3/75/PR8 H3N2, A/Udorn/72 H3N2 and A/Vietnam/1194/04/PR8H5N1.

The present inventor has determined that binding of an agent of formula A to haemagglutinin inhibits the ability of a virus, in particular an influenza virus to infect a cell, enabling the agent to act in a prophylactic manner to minimise a subject's infection by and risk of infection by the virus, in particular influenza.

Accordingly, a first aspect of the present invention provides an agent of formula A comprising a peptide having an amino acid sequence X₁-X₂-X₃-X₄-X₅-X₆, (SEQ ID NO 1) wherein

X₁ can be phenylalanine, isoleucine or tryptophan;
X₂ can be leucine or phenylalanine or alanine;
X₃ can be tyrosine or valine;
X₄ can be leucine, phenylalanine or isoleucine;
X₅ can be phenyalanine or alanine; and
X₆ can be valine, arginine or tyrosine,
or a fragment or variant of the peptide, wherein said agent or peptide fragment or variant is capable of specifically binding to haemagglutinin, to inhibit the binding of a virus having haemagglutinin on its surface, for example influenza, to a cell, for use in the treatment of a virus, for example influenza.

Thus, the invention provides a method for treating a pathology associated with a virus which includes haemagglutinin on its surface, in particular the pathology of influenza. The method is practiced by administering to a subject, for example a human a therapeutic amount of one or more agents of the invention thereby to treat an established viral infection of said virus or prophylactically treat a viral infection, in particular influenza. In embodiments the agent inhibit or block the haemagglutinin receptor on the virus.

In embodiments, the agent can bind to haemagglutinin on influenza to inhibit the binding of influenza to a cell. In such embodiments, the agent can be used for the treatment of influenza, in particular the prophylactic treatment of influenza. A suitable functional test which allows the binding of an agent to haemagglutinin to be determined is an ELISA based assay, for example the influenza haemagglutinin binding assay (ELISA) as described herein or a suitable modification of this ELISA using a different respective virus with haemagglutinin on its surface.

In embodiments an agent of formula A can comprise a peptide having at least 4, at least 5, at least 6 amino acids from the C or N terminus of

| SEQ ID NO 11 | |
|---|---|
| FP2 | WLVFFVIAYFAR |
| or | |
| SEQ ID NO 18 | |
| FP1 | WLVFFVIFYFFR |

characterised in that the peptide can specifically bind to haemagglutinin.

In yet further embodiments the agent of formula A can be at least 4, 5, or 6 consecutive amino acids of FP2 or FP1 characterised in that the peptide can specifically bind to haemagglutinin. In yet further embodiments the agent of formula A can be at least 4, 5, or 6 consecutive amino acids of FP2 characterised in that the peptide can specifically bind to haemagglutinin.

In embodiments an agent of formula A can be a peptide that does not comprise the C-terminal 1, 2, 3, 4, 5, 6, 7 amino acids of FP2 or FP1, in particular FP2 characterised in that the peptide can specifically bind to haemagglutinin.

In embodiments an agent of formula A can be a peptide that does not comprise the N-terminal 1, 2, 3, 4, 5, 6, 7 amino acids of FP2 or FP1, in particular FP2, characterised in that the peptide can specifically bind to haemagglutinin.

In embodiments, at least 1, 2, or 3 amino acids of a peptide fragment having at least 4, at least 5, at least 6 consecutive amino acids of FP2 or FP1 may be conservatively substituted with another amino acid characterised in that the peptide can specifically bind to haemagglutinin. In embodiments, the agent of formula A can have at least 4 consecutive amino acids of the peptide WLVFFVIAYFAR (FP2) (SEQ ID NO 11) or can be a variant of the peptide FP2 wherein the peptide variant comprises between 1 and 3 amino acids conservatively substituted with another amino acid characterised in that the peptide can specifically bind to haemagglutinin. It will be understood that said variant may include further peptidomimetic modifications as discussed herein, for example to increase the stability of the peptide.

By conservative substitution is meant the replacement of one amino acid with another that is biologically and/or chemically similar, as would be understood by those of skill in the art.

As exemplified by the particular sequences FP1, FP2, FP3, FP4, FP7, FP8, and FP9 discussed herein, an agent of the invention may be a peptide comprising at least 4, at least 5 at least 6 amino acids which can form a coiled or alpha helical structure with the amino acid sequence of the peptide being generally aliphatic or hydrophobic. Without wishing to be bound by theory the three dimensional structure of such a peptide may provide at least one hydrophobic region or patch on one side of a coiled structure, for example an alpha-helix. Alternatively, the three dimensional structure may provide at least two hydrophobic regions on opposite faces of a coiled structure such as an alpha helix or on the same face of a coiled structure wherein the hydrophobic regions are spaced apart. In embodiments an agent of the invention may be a peptide with an amino acid sequence comprising

(SEQ ID NO 2)
FFVIFY
(SEQ ID NO 5)
WLVFFV,
or
(SEQ ID NO 16)
FFVIAYFAR
(SEQ ID NO 22)
FFVIAY,
(SEQ ID NO 23)
IAYFAR

In particular embodiments, an agent of the invention, in particular SEQ ID NO 2, 5, 16, 22, or 23 may include a peptide or mimetic thereof wherein one two, or three phenylalanine amino acid residues are substituted with a tyrosine. Further, an agent of the invention, may include a peptide or mimetic thereof wherein the N-terminal amino acid is provided as a D amino acid.

Without wishing to be bound by theory, the inventor believes that advantageous agents, peptides or variants of a peptide of the invention may promote adoption of a coiled structure, in particular an alpha helical structure.

It is considered that such a coiled structure is advantageous in enabling the peptide to interact with virus. Structure prediction may be implemented using suitable computer programs, for example GOLD-Protein-Ligand Docking (University of Sheffield, GlaxoSmithKline plc and CCDC) and ExPASy ProtParam program, PEP FOLD mobile program. In an embodiment when the peptide provided is FFVIFY (SEQ ID NO 2), RRKK (SEQ ID NO 3) can be provided at the N terminal end of the peptide to provide RRKKFFVIFY (SEQ ID NO 4) which is believed to adopt a coiled structure. The addition of amino acid sequences, such as for example RRKK, may advantageously improve the solubility of the peptide in an aqueous solution for example by reducing hydrophobicity and allow higher doses of the peptide to be administered in vitro and in vivo. As will be appreciated, other hydrophilic peptide sequences, for example Arginine×9 which can allow increased solubility of a peptide can be conjoined to a peptide of the invention.

In embodiments when the peptide provided has the amino acid sequence WLVFFV (SEQ ID NO 5) or FFVIFY (SEQ ID NO 2) it has been found to be advantageous to provide RRKK (SEQ ID NO 3) at the C terminal end of the peptide to provide WLVFFVRRKK (SEQ ID NO 6) and FFVIFYRRKK (SEQ ID NO 7) respectively. These peptides are believed to adopt a coiled structure and, in addition have a reduced hydrophobicity relative to SEQ ID NO 5 and SEQ ID NO 2.

In embodiments a peptide of the invention can comprise an amino acid sequence selected from at least one of SEQ ID NO 8, 9, 2, and 10 (as described by Table a) or a fragment or variant of an amino acid sequence as provided by SEQ ID No 8, 9, 2 and 10 capable of specifically binding to haemagglutinin of a virus, inhibiting the binding of the virus to a cell. In embodiments a peptide of the invention may bind specifically to haemagglutinin of influenza and inhibit the binding of influenza to a cell. The binding of an agent of the invention to a cell can be determined using an ELISA assay, as set out herein with reference to influenza, or an appropriate virus. Alternatively a functional test to observe a reduced number of plaques caused by a virus, or a reduced clinical sign of the virus can be used to assess binding to haemagglutinin.

TABLE a

| X1 | X2 | X3 | X4 | X5 | X6 | SEQ ID NO |
|---|---|---|---|---|---|---|
| I | F | Y | F | F | R | 8 |
| W | L | V | F | F | V | 9 |
| F | F | V | I | F | Y | 2 |
| I | A | Y | F | A | R | 10 |

wherein the standard single letter code symbols are utilised, for example W is tryptophan, L is leucine, V is valine, F is phenylalanine, Y is tyrosine, R is arginine, I is isoleucine and A is alanine.

An alanine mutant of SEQ ID NO 8 is shown (SEQ ID NO 10). The substitution of phenylalanine with alanine has been determined to cause the peptide to be an effective inhibitor of H3 and H5 viruses as well as H1 viruses. It may be expected that other substitutions of small aliphatic amino acids at these positions would also be effective. In particular embodiments a peptide of the invention can comprise an amino acid sequence selected from at least one of

| SEQ ID NO 11 | |
|---|---|
| FP2 | WLVFFVIAYFAR |
| SEQ ID NO 12 | |
| FP3 | WLVFFVIFYFFRRRKK |
| SEQ ID NO 13 | |
| FP4 | RRKKWLVFFVIYFFR |
| SEQ ID NO 6 | |
| FP8 | WLVFFVRRKK |
| SEQ ID NO 7 | |
| FP9 | FFVIFYRRKK |
| SEQ ID NO 14 | |
| FP10 | IVWFYLFRFFVF |
| SEQ ID NO 15 | |
| FP11 | FFVIAYRRKK |
| SEQ ID NO 16 | |
| FP12 | FFVIAYFAR |
| SEQ ID NO 17 | |
| FP13 | FFVIAYFARRRKK |

or a fragment or variant of an amino acid sequence as provided by SEQ ID No 11, 12, 13, 6, 7, 14, 15, 16 or 17 capable of binding specifically to haemagglutinin of a virus and inhibiting the binding of the virus to a cell in particular to haemagglutinin of influenza and inhibiting the binding of influenza to a cell.

In embodiments a peptide can comprise an amino acid sequence selected from

| SEQ ID NO 11 | |
|---|---|
| FP2 | WLVFFVIAYFAR, |
| SEQ ID NO 12 | |
| FP3 | WLVFFVIFYFFRRRKK, |
| SEQ ID NO 13 | |
| FP4 | RRKKWLVFFVIYFFR, |
| SEQ ID NO 14 | |
| FP10 | IVWFYLFRFFVF |

or a fragment or variant of an amino acid sequence as provided by SEQ ID No 11 to 14 capable of binding specifically to haemagglutinin of a virus and inhibiting the binding of the virus to a cell in particular to haemagglutinin of influenza and inhibiting the binding of influenza to a cell.

In embodiments a peptide of the invention can comprise an amino acid sequence selected from

| SEQ ID NO 12 | |
|---|---|
| FP3 | WLVFFVIFYFFRRRKK |
| SEQ ID NO 11 | |
| FP2 | WLVFFVIAYFAR |
| SEQ ID NO 13 | |
| FP4 | RRKKWLVFFVIYFFR |

or a fragment or variant of an amino acid sequence as provided by SEQ ID No 11 to 13 capable of binding specifically to haemagglutinin of a virus and inhibiting the binding of the virus to a cell in particular to haemagglutinin of influenza and inhibiting the binding of influenza to a cell.

In embodiments a peptide of the invention can comprise an amino acid sequence selected from

| SEQ ID NO 12 | |
|---|---|
| FP3 | WLVFFVIFYFFRRRKK, |
| SEQ ID NO 13 | |
| FP4 | RRKKWLVFFVIYFFR, |

or a fragment or variant of an amino acid sequence as provided by SEQ ID No 12 or 13 capable of binding specifically to haemagglutinin of a virus and inhibiting the binding of the virus to a cell in particular to haemagglutinin of influenza and inhibiting the binding of influenza to a cell.

In embodiments a peptide of the invention can comprise an amino acid sequence WLVFFVIAYFAR (SEQ ID NO 11) or a fragment or variant of SEQ ID NO 11 capable of binding specifically to haemagglutinin. In embodiments a peptide of the invention can independently comprise any of SEQ ID NO 1 to 18, 22 or 23, in particular 2 to 17, 22 or 23 for use in the treatment of influenza.

In embodiments, SEQ ID NO 11 can be modified to include RRKK or KKKK or another hydrophilic peptide at the N or C terminal. In particular embodiments SEQ ID NO 11 can be provided as a truncated peptide having a sequence FFVIAYFAR (SEQ ID NO 16). In alternative embodiments, SEQ ID NO 11 can be provided as a ninemer with wherein 2, 3, 4, 5, 6, 7, or 8 amino acids are conservatively replaced.

In embodiments, an agent of the present invention can be between 6 to 20 amino acids in length, preferably 6 to 15 amino acids in length, more particularly 6, 7, 8, 9, 10, 11, 12, 13, 14, 15 amino acids in length.

In embodiments of the invention a peptide of the invention can consist of an amino acid sequence selected from

| SEQ ID NO 11 | |
|---|---|
| FP2 | WLVFFVIAYFAR, |
| SEQ ID NO 12 | |
| FP3 | WLVFFVIFYFFRRRKK, |
| SEQ ID NO 13 | |
| FP4 | RRKKWLVFFVIYFFR, |
| SEQ ID NO 6 | |
| FP8 | WLVFFVRRKK, |
| SEQ ID NO 7 | |
| FP9 | FFVIFYRRKK, |
| SEQ ID NO 14 | |
| FP10 | IVWFYLFRFFVF, |
| SEQ ID NO 15 | |
| FP11 | FFVIAYRRKK, |
| SEQ ID NO 16 | |
| FP12 | FFVIAYFAR |
| SEQ ID NO 17 | |
| FP13 | FFVIAYFARRRKK |

or a fragment or variant of an amino acid sequence as provided by SEQ ID No 11, 12, 13, 6, 7, 14, 15, 16, and 17 capable of binding specifically to haemagglutinin of a virus and inhibiting the binding of the virus to a cell in particular to haemagglutinin of influenza and inhibiting the binding of influenza to a cell.

In further embodiments a peptide of the invention can consist of an amino acid sequence selected from at least one of

| SEQ ID NO 11 | |
|---|---|
| FP2 | WLVFFVIAYFAR, |
| SEQ ID NO 12 | |
| FP3 | WLVFFVIFYFFRRRKK, |
| SEQ ID NO 13 | |
| FP4 | RRKKWLVFFVIYFFR, |
| SEQ ID NO 14 | |
| FP10 | IVWFYLFRFFVF |

or a fragment or variant of an amino acid sequence as provided by SEQ ID No 11, 12, 13, or 14 capable of binding specifically to haemagglutinin of a virus and inhibiting the binding of the virus to a cell in particular to haemagglutinin of influenza and inhibiting the binding of influenza to a cell.

In yet further embodiments a peptide of the invention can consist of an amino acid sequence selected from

| SEQ ID NO 11 | |
|---|---|
| FP2 | WLVFFVIAYFAR, |
| SEQ ID NO 13 | |
| FP4 | RRKKWLVFFVIYFFR, |
| SEQ ID NO 12 | |
| FP3 | WLVFFVIFYFFRRRKK |

or a fragment or variant of an amino acid sequence as provided by SEQ ID No 11, 12, 13, capable of binding specifically to haemagglutinin of a virus and inhibiting the binding of the virus to a cell in particular to haemagglutinin of influenza and inhibiting the binding of influenza to a cell.

In embodiments a peptide of the invention can consist of an amino acid sequence WLVFFVIAYFAR (SEQ ID NO 11).

It should be understood that combinations of agents of formula A can be provided to a subject simultaneously or sequentially.

In a second aspect of the present invention there is provided an agent, in particular a peptide of formula A, as described according to the first aspect, for use in the prevention or prophylactic treatment of virus infection in a subject, in particular influenza infection in a subject.

Suitably, an agent of formula A, as described according to the first aspect may be used in the prevention or prophylactic treatment of virus infection in a subject wherein the virus is selected from measles virus, mumps virus, parainfluenza virus, respiratory syncytial virus (RSV), rubella virus, rabies virus, nipah virus, hendra virus, canine distemper virus, phocine distemper virus, rinderpest virus, Newcastle disease virus, metapneumovirus, or sendai virus.

In particular, the present invention relates to an agent of formula A for use in the prophylactic treatment of influenza.

In particular embodiments an agent of formula A, as described according to a first aspect of the invention, may be used in the prevention or prophylactic treatment of influenza of type A, more particularly influenza types H1N1, H3N2, H5N1 and H7N1, more particularly influenza A/WSN/33 H1N1, A/PR8/34 H1N1, A/England/195/09/PR8H1N1, A/Victoria/3/75/PR8H3N2, A/Udorn/72 H3N2 and A/Vietnam/1194/04/PR8H5N1.

In particular embodiments an agent of formula A can be selected from any one of SEQ ID NOs 1 to 17, in particular SEQ ID NOs 11, 12, 13, or 14, more particularly SEQ ID NOs 11, 12, or 13, most particularly SEQ ID NO 11 or 13.

As will be appreciated, a subject may have never been exposed to a virus such as influenza or may have been exposed to a different strain or type of virus such as a different type of influenza. Whilst a subject may have previously been infected by an influenza virus strain, should the subject be exposed to a different strain, the subject's immune system will mount a new immune response to the different strain. An agent, for example a peptide, of formula A may be provided to a subject prior to the exposure of the subject to a virus expressing haemagglutinin on its surface, for example influenza, for a first time or to virus, for example influenza, of a different strain, to minimise the subject's risk of infection.

Whilst some peptides of the present invention are capable of binding to kinases and acting to prevent the autophosphorylation and signalling of such kinases, for example kinases involved in mediating the inflammatory response in subjects infected by influenza, the present inventor has surprisingly determined a number of agents, in particular peptides, of the present invention which can bind to haemagglutinin to inhibit viral entry to a call, for example to inhibit influenza entry into a cell, but which are not capable of modulating kinase autophosphorylation. In embodiments such agents/peptides can be provided to subjects at risk or at increased risk of being infected a virus expressing haemagglutinin on its surface, for example with influenza, as a preventative treatment to minimise the risk of infection, for example of influenza infection. In embodiments, the use of peptides which are capable of modulating the inflammatory response in a subject can be excluded. In embodiments, the use of peptides which are capable of modulating kinase autophosphorylation are excluded. In specific embodiments, the use of WLVFFVIFYFFR (SEQ ID NO 18) can be excluded.

In particular embodiments, peptides comprising an amino acid sequence of the present invention in which the amino acids at position X₂ or X₅ are substituted with alanine, in particular peptides with the amino acid sequence WLVFFVIAYFAR (SEQ ID NO 11) have been determined to block the immunomodulatory activity of the peptide, whilst not affecting the anti-viral activity of the peptide against influenza. Without wishing to be bound by theory, it is considered that providing X₂ and/or X₅ as alanine amino acids increases the inhibition of virus infection provided in relation to H3N2 viruses as well as H1N1 and H5N1.

The present invention provides for the use of a peptide of the invention as disclosed herein in the manufacture of a medicament for the treatment of influenza or prevention of infection by influenza. There is further provided a method of treating influenza or preventing infection by influenza of a subject comprising administering to said subject an effective amount of an agent, for example a peptide of the invention as disclosed herein.

An agent of the invention, for example a peptide, or a substance or composition comprising such an agent may be administered alone or in combination with other treatments, either simultaneously or sequentially dependent upon the condition to be treated. Such combinations may be selected based on, for example, the conditions to be treated, the reactive activities of the ingredients and pharmaceutical properties of the combinations. For example, an agent of the invention may be combined with other antivirals such as amantidine, rimantadine, ribavirin, neuraminidase inhibitors, mucolytics, expectorants, bronchialdilators, antibiotics or analgesics.

According to a further aspect of the present invention there is provided a method of therapy comprising the step of administering a therapeutic amount of an agent of the present invention to a subject in need thereof. Suitably a subject may be at risk of or be infected with a virus with haemagglutinin on its surface, for example at risk or be infected with influenza.

Whatever agent or peptide of the invention used in a method of medical treatment of the present invention, administration is preferably in a “prophylactically effective amount” or a “therapeutically effective amount” (as the case may be), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

In embodiments, an agent, for example a peptide, of the invention can be provided to a mammal. In particular embodiments, an agent, for example a peptide can be provided to at least one of a human(s), a pig(s), a horse(s), a cat(s) an avian, for example poultry. In specific embodiments, an agent of the invention, for example a peptide of the invention can be provided to a human.

Targeting therapies may be used to deliver the agent of the invention more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

In determining which agents, for example peptides, of the invention act to confer anti-viral activity, the inventor has determined peptides of the invention with novel amino acid sequences. These peptides per se are considered to form a further aspect of the invention.

According to a third aspect of the present invention there is provided an agent of formula A as disclosed herein. In embodiments, the use of peptides which are capable of modulating the inflammatory response in a subject can be excluded. In embodiments, the use of peptides which are capable of modulating kinase autophosphorylation are excluded. In specific embodiments, the use of WLVFFVIFYFFR (SEQ ID NO 18) can be excluded.

In embodiments, there is provided an agent of formula A comprising or consisting of a peptide having an amino acid sequence selected from any one of SEQ ID NOs 1 to 17, 21, 22, or 23. In embodiments there is provided an agent of formula A comprising or consisting of a peptide having an amino acid sequence having at least 4 consecutive amino acids of WLVFFVIAYFAR (FP2) or a variant of the peptide wherein the peptide variant comprises between 1 and 3 amino acids conservatively substituted with another amino acid characterised in that the peptide can specifically bind to haemagglutinin.

In embodiments there is provided an agent of formula A comprising or consisting of a peptide having an amino acid sequence of any one of SEQ ID NOs 1 to 17. In embodiments there is provided an agent of formula A comprising or consisting of a peptide having an amino acid sequence SEQ ID NO 22 or 23 or 11. In embodiments there is provided a peptide comprising an amino acid sequence selected from

| SEQ ID NO 11 | |
|---|---|
| FP2 | WLVFFVIAYFAR, |
| SEQ ID NO 7 | |
| FP9 | FFVIFYRRKK, |
| SEQ ID NO 14 | |
| FP10 | IVWFYLFRFFVF, |
| SEQ ID NO 2 | |
| | FFVIFY, |
| SEQ ID NO 15 | |
| FP11 | FFVIAYRRKK, |
| SEQ ID NO 16 | |
| FP12 | FFVIAYFAR, |
| SEQ ID NO 17 | |
| FP13 | FFVIAYFARRRKK |

or a variant or fragment of the peptide wherein said fragment is capable of specifically binding to haemagglutinin, particularly haemagglutinin as provided by influenza. In embodiments, the peptide can bind to haemagglutinin such that entry of a virus having haemagglutinin on its surface into a cell is minimised.

In embodiments an agent of formula A can comprise a peptide having at least 4, at least 5, at least 6 amino acids from the C or N terminus of FP2

SEQ ID NO 11
WLVFFVIAYFAR

characterised in that the peptide can specifically bind to haemagglutinin.

In alternative embodiments an agent of formula A can be a peptide that does not comprise the C-terminal 1, 2, 3, 4, 5, 6, 7 amino acids of FP2 characterised in that the peptide can specifically bind to haemagglutinin.

In still further embodiments an agent of formula A can be a peptide that does not comprise the N-terminal 1, 2, 3, 4, 5, 6, 7 amino acids of FP2 characterised in that the peptide can specifically bind to haemagglutinin.

In yet further embodiments the agent of formula A can be at least 4, 5, or 6 consecutive amino acids of FP2.

In embodiments, at least 1, 2, or 3 amino acids of a peptide fragment having at least 4, at least 5, at least 6 consecutive amino acids of FP2 may be conservatively substituted with another amino acid characterised in that the peptide can specifically bind to haemagglutinin.

In embodiments an agent of the invention may be a peptide with an amino acid sequence comprising

(SEQ ID NO 2)
FFVIFY
(SEQ ID NO 5)
WLVFFV,
or
(SEQ ID NO 16)
FFVIAYFAR
(SEQ ID NO 22)
FFVIAY,
(SEQ ID NO 23)
IAYFAR

In particular embodiments, an agent of the invention, in particular SEQ ID NO 2, 5, 16, 22, or 23 may include a peptide or mimetic thereof wherein one or more phenylalanine amino acid residues are substituted with a tyrosine. Further, an agent of the invention, may include a peptide or mimetic thereof wherein the N-terminal amino acid is provided as a D amino acid.

In embodiments the peptide can consist of an amino acid sequence selected from

| SEQ ID NO 11 | |
|---|---|
| FP2 | WLVFFVIAYFAR, |
| SEQ ID NO 7 | |
| FP9 | FFVIFYRRKK, |
| SEQ ID NO 14 | |
| FP10 | IVWFYLFRFFVF, |
| or | |
| SEQ ID NO 2 | |
| | FFVIFY. |

In embodiments, the peptide can consist of an amino acid sequence selected from

| SEQ ID NO 15 | |
|---|---|
| FP11 | FFVIAYRRKK, |
| SEQ ID NO 16 | |
| FP12 | FFVIAYFAR |
| or | |
| SEQ ID NO 17 | |
| FP13 | FFVIAYFARRRKK. |

The invention also provides pharmaceutical preparations comprising an agent of formula A together with a pharmaceutically acceptable excipient. The peptides of the invention may be provided in purified, synthetic or recombinant form.

Instead of administering such an agent, for example a peptide, directly, it may be produced in a target cells by expression from an encoding nucleic acid introduced into the cell, e.g. from a viral vector. The vector may be targeted to the specific cell(s) to be treated, or it may contain regulatory elements which are switched on more or less selectively by the target cell(s).

According to a fourth aspect of the present invention there is provided a nucleic acid sequence which can encode a peptide as described herein, in particular an amino acid sequence of the third aspect of the invention.

According to a fifth aspect of the present invention there is provided an expression construct comprising a nucleic acid of the fourth aspect of the invention, in particular a nucleic acid of the fourth aspect of the invention which can encode an amino acid sequence of a peptide disclosed herein, in particular a peptide of the third aspect of the invention and a promoter region operably linked to the nucleic acid sequence. By operably linked is meant a juxtaposition of the components described such that they are in a relationship which allows them to function in their intended manner. The promoter region can include regulatory elements which are functional in an intended host cell in which the expression construct is to be expressed. Expression constructs can be provided using techniques as known in the art and utilising promoters and regulatory elements as known in the art for use in a host cell of choice.

In preferred embodiments, the regulatory elements controlling expression can be inducible on virus infection.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically. Nucleic acid encoding the active agent may thus be used in methods of gene therapy, for instance in treatment of individuals, e.g. with the aim of preventing or curing (wholly or partially) a viral infection, for example influenza. This may be particularly useful to provide an agent of the invention to for example a chicken or other poultry or a domesticated animal such as a pig. The poultry or domesticated animal would therefore be less susceptible to viral infection, for example influenza infection. This could be advantageous in reducing the reservoir of for example avian influenza virus, which can lead to pandemic virus outbreaks. A transgenic animal able to express a peptide of the invention provides a separate aspect of the present invention.

A variety of vectors, both viral vectors and plasmid vectors, are known in the art, see U.S. Pat. No. 5,252,479 and WO 93/07282. As an alternative to the use of viral vectors in gene therapy other known methods of introducing nucleic acid into cells including mechanical techniques such as microinjection, transfer mediated by liposomes and receptor-mediated DNA transfer may be utilised.

A further use of the agents of the invention is in the prevention and control of viral infection through engineering of constituents of the indigenous microflora to constitutively express the agents. Methods which serve as an example methodology for such use can be the engineering of Lactobacillus from the human vaginal microflora to express peptide inhibitors of HIV-1 entry and fusion (Pusch O, Kalyanaraman R, Tucker L D, Wells J M, Ramratnam B, Boden D. 2006. An anti-HIV microbicide engineered in commensal bacteria: secretion of HIV-1 fusion inhibitors by lactobacilli. AIDS 20:1917-22; and Liu J J, Reid G, Jiang Y, Turner M S, Tsai C C. 2007. Activity of HIV entry and fusion inhibitors expressed by the human vaginal colonizing probiotic Lactobacillus reuteri RC-14. Cell. Microbiol. 9:120-30), decoy receptors (Chang T L, Chang C H, Simpson D A, Xu Q, Martin P K, Lagenaur L A, Schoolnik G K, Ho D D, Hillier S L, Holodniy M, Lewicki J A, Lee P P. 2003. Inhibition of HIV infectivity by a natural human isolate of Lactobacillus jensenii engineered to express functional two-domain CD4. Proc. Natl. Acad. Sci. U.S. A. 100:11672-7) or chemokines that antagonize viral replication (Vangelista L, Secchi M, Liu X, Bachi A, Jia L, Xu Q, Lusso P. 2010. Engineering of Lactobacillus jensenii to secrete RANTES and a CCR5 antagonist analogue as liveHIV-1 blockers. Antimicrob. Agents Chemother. 54:2994-3001).

Thus, a further aspect of the present invention comprises an indigenous microflora bacteria or cell engineered to constitutively or inducibly express an agent of the present invention. In embodiments the bacteria can be Lactobacillus. Suitably an agent may be any agent discussed herein, in particular an agent selected from any one of SEQ ID NO 1 to 17. Further, there is provided a method of modifying such bacteria to provide an agent of the present invention. Such microflora bacteria could be provided to the subject as a probiotic culture.

Bacteria expressing such an agent can be considered to be a bioshield. Such tioshields' may be effective in control of a plethora of infectious diseases; however the host-specific nature of HIV-1 has precluded analysis of protective efficacy to date. Expression of peptides of the invention in avian intestinal Lactobacilli would serve as a valuable proof-of-potential of the approach and facilitate access to a vast market. Recent studies have identified Lactobacilli that persist effectively in the chicken intestines (Stephenson D P, Moore R J, Allison G E. 2010. Lactobacillus strain ecology and persistence within broiler chickens fed different diets: identification of persistent strains. Appl. Environ. Microbiol. 76:6494-503) and which are suitable for expression of heterologous antigens in poultry Stephenson D P, Moore R J, Allison G E. 2011. Transformation of, and heterologous protein expression in, Lactobacillus agilis and Lactobacillus vaginalis isolates from the chicken gastrointestinal tract. Appl. Environ. Microbiol. 77:220-8; and Mota R M, Moreira J L, Souza M R, Horta M F, Teixeira S M, Neumann E, Nicoli J R, Nunes A C. 2006. Genetic transformation of novel isolates of chicken Lactobacillus bearing probiotic features for expression of heterologous proteins: a tool to develop live oral vaccines. BMC Biotechnol. 6:2].

An example of a proposed methology in such an embodiment utilising the determinations of the present inventor is
1. Engineer avian-adapted Lactobacilli to express at least one agent, in particular a peptide of the invention in secreted and surface-anchored forms.
2. Evaluate the ability of the agent, for example a peptide, of the invention produced by Lactobacilli to interfere with virus entry, for example influenza virus entry, and propagation in vitro.
3. Evaluate the ability of Lactobacilli to persist at sites of viral replication in poultry and produce the peptide of the invention in vivo.
4. Evaluate if the peptide-expressing Lactobacilli protect chickens against experimental viral infection, for example avian influenza virus infection.
5. Optimise properties of the peptide and vector system to enhance the magnitude and duration protection.

Probiotic bacteria have gained wide public acceptance in recent years and are commonly used in poultry production in formulations such as Avigaurd® and Broilact®. They can be administered in drinking water, are inexpensive to culture and pose negligible threats to animal or human health. Variants of indigenous flora engineered to express beneficial molecules, such as an agent of the invention, may prove less controversial that live-attenuated derivatives of pathogens or transgenesis as delivery systems. Once developed, such vector systems could be adapted to target a plethora of pathogen-host combinations and processes

A peptide of the invention may be readily prepared using standard techniques known in the art including chemical synthesis and genetic engineering. A peptide of the present invention can include the specific peptides exemplified herein as well as variant peptides thereof which may be, for example, longer or shorter than the peptides illustrated. For example, a person of skill in the art could readily make peptides having from 1 to about 15 or more amino acids added to one or both ends of a peptide, more preferably wherein 1, 2, 3, 4, 5, 6, 7, 8, or 9 amino acids are added to one or both ends of a peptide of the invention. Examples of peptides, having amino acids added to one or both ends of a core peptide contemplated within the scope of the present invention are

(SEQ ID NO 15)
FP11 FFVIAY(RRKK)
(SEQ ID NO 16)
FP12 FFVIAYFAR +/− RRKK
(SEQ ID NO 17)
FP13 FFVIFYFFR +/− RRKK
(SEQ ID NO 19)
FP14 IFYFFR(RRKK)-FP7 with RRKK at C terminus

Similarly a person skilled in the art could readily prepare peptides in which about 1, 2, 3, 4 or 5 amino acids are removed from one or both ends of a peptide of the present invention. The present invention includes, but is not limited to, variant peptides, wherein such peptides can have 5, 6, 7, 8, 9, 10, 11, 12, 13, 14, 15, 16, 17, 18, 19, and 20 amino acids longer or shorter in length than the illustrated peptides wherein the variant peptides (lengthened or truncated) still retain or show enhanced activity in blocking binding of viral entry into a cell, for example of the influenza virus to the cell. In particular, such peptides should be able to specifically bind to haemagglutinin and inhibit viral entry into the cell. Typically such peptides may be at least five or six amino acids in length. Without wishing to be bound by theory, the inventor considers that addition of hydrophilic sequences to the core peptides discussed herein may alter the secondary structure of the peptides such that the peptides are provided as a coiled structure.

By inhibition of viral entry into the cell is meant to be at least a 20% reduction in viral entry in the presence of a peptide or agent of the invention in comparison to a control in which the peptide or agent is not present, more preferably a reduction of at least 30%, at least 40%, at least 50% at least 60%, at least 70%, at least 80%, at least 90% in viral infection when compared to a control cell. Suitably a test control cell may be a Madin-Darby canine kidney cell or a lung epithelial cell. The influence of a peptide or an agent on viral entry may be tested experimentally by infecting a test cell with a known number of plaque forming units in the absence of a test peptide of the invention or agent and in the presence of increasing quantities of peptide or agent and determining the degree of reduction of infection with respect to the amount of agent present. In embodiments a peptide or agent which resulted in a reduction of infection of at least 50% would be further characterised.

Variant peptides can include peptides having a conservative substitution of the amino acids specifically provided in the peptide amino acid sequences specifically provided herein. As would be understood by those of skill in the art, amino acids can generally be categorised in the following classes, non-polar, un-charged polar, basic and acidic. For example nonpolar amino acids include alanine, leucine, isoleucine, valine, proline, phenylalanine, tryptophan and methionine, uncharged polar amino acids include glycine, serine, threonine, cysteine, tyrosine, asparagine, and glutamine. Basic amino acids include arginine, lysine and histidine and acidic amino acids include aspartic acid and glutamic acid. Conservative substitution whereby an amino acid of one class is replaced with another amino acid of the same class are considered to fall within the scope of the invention, provided said agent can inhibit the binding of a virus to a cell, for example influenza to a cell and the agent can bind to haemagglutinin. In specific embodiments, 1, 2, 3, 4, or 5 amino acids can be substituted.

Variants can include multimers of the peptides of the invention, in particular of SEQ ID NO 1 to 17, 22 or 23, for example at least two, at least 3, at least 4, or at least 5 of SEQ ID NO 1 to 17, 22 or 23 can be conjoined to each other. Such multimers may be used to prepare a monomeric peptide by preparing a multimeric peptide that includes the monomeric unit (for example any one of SEQ ID NO 1 to 17), and a cleavable site (i.e., an enzymatically cleavable site) between such monomeric units, and then cleaving the multimer to yield a desired monomer. The use of multimers may advantageously increase the binding affinity of the peptide to haemagglutinin. The multimers can be homomers or heteromers. For example, a homomer can include only peptides of the invention having an identical amino acid sequence, whilst a heteromer can include one or more heterologous peptides of the invention.

The multimers may be the result of hydrophobic, hydrophilic, ionic and/or covalent associations and/or may be indirectly linked by, for example, liposome formation. In one embodiment, covalent association can be the consequence of chemical or recombinant manipulation. Alternatively, such covalent associations can involve one or more amino acid residues contained in a heterologous peptide sequence of the invention. In another embodiment, two or more polypeptides described herein are joined through peptide linkers. Proteins comprising multiple peptides separated by peptide linkers can be produced using conventional recombinant DNA technology.

Multimers may also be prepared by fusing the peptides of the invention to a leucine zipper or isoleucine zipper polypeptide sequence. Among the known leucine zippers are naturally occurring peptides and derivatives thereof that dimerize or trimerize. Recombinant fusion proteins comprising a polypeptide described herein fused to a polypeptide sequence that dimerizes or trimerizes in solution can be expressed in suitable host cells, and the resulting soluble multimeric fusion protein can be recovered from the culture supernatant using techniques known in the art.

The multimers may also be generated using chemical techniques known in the art. For example, peptides desired to be contained in the multimers described herein may be chemically cross-linked using linker molecules and linker molecule length optimization techniques known in the art. Additionally, the multimers can be generated using techniques known in the art to form one or more inter-molecule cross-links between cysteine residues located within the sequence of the peptides desired to be contained in the multimer

In embodiments, a variant can also include a non-peptide compound(s) and/or a non-natural amino acid(s) that mimic the function of the amino acid sequences of the present invention and/or mimic the tertiary structure or activity of an agent/peptide of the invention. Such mimic, mimetic or peptidomimetic variants that can include non-peptide “small molecules” which are often preferred for in vivo pharmaceutical use, retain the functional and binding properties of amino acid sequences of the invention. The skilled person would be aware of methods for preparing such mimetics or peptidomimetics based on the amino acid sequences provided.

In embodiments, variants can include peptides wherein non-natural amino acids can be substituted for the amino acids of an agent of the invention, provided the agent having the substituted amino acids retains the ability to inhibit the binding of a virus to a cell, for example influenza to a cell and to specifically bind to haemagglutinin. Examples of non natural amino acids include, but are not limited to: ornithine, citrulline, hydroxyproline, homoserine, phenylglycine, taurine, iodotyrosine, 2,4-diaminobutyric acid, [alpha]-amino isobutyric acid, 4-aminobutyric acid, 2-amino butyric acid, [gamma]-amino butyric acid, [epsilon]-amino hexanoic acid, 6-amino hexanoic acid, 2-amino isobutyric acid, 3-amino propionic acid, norleucine, norvaline, sarcosine, homocitrulline, cysteic acid, [tau]-butylglycine, [tau]-butylalanine, phenylglycine, cyclohexylalanine, [beta]-alanine, fluoro-amino acids, designer amino acids such as [beta]-methyl amino acids, C-methyl amino acids, N-methyl amino acids, and amino acid analogues in general. Non-natural amino acids also include amino acids having derivatized side groups. A variant can also include a pharmaceutically acceptable salt of a peptide.

In embodiments, an agent of the invention may have an amino acid sequence of the invention wherein one or more of the amino acids may be provided in a D amino acid form. In particular embodiments, the first amino acid of the peptide may be provided as a D amino acid to advantageously stabilise the peptide. Alternatively, molecules which resemble peptides can be provided wherein the amino aids or amino acid analogs are not connected via natural peptide linkages, for example such linkages can include ester, thioester, thioamide, retroamide, reduced carbonyl, dimethylene and ketomethylene bonds and others as would be known in the art. Peptidomimetics of the peptides of the invention may also have amino acids which have been chemically modified by phosphorylation, sulfonation, biotinylation, or the addition or removal of other moieties. Amino acid analogs and peptide mimetics often have enhanced or desirable properties such as more economical production, greater chemical stability, enhanced pharmacological properties (half life, potency, efficacy etc) or a greater ability to cross biological barriers, for example the gut.

Variants of an agent of the invention or variants for use in the present invention further include reverse- or retro-analogues of peptides of the invention or their synthetic derivatives. Reverse peptides are produced by reversing the amino acid sequence of the peptide. It is believed that such reverse-peptides retain the same general three-dimensional structure as the parent peptide except for the conformation around internal protease-sensitive sites and the characteristics of the N- and C-termini. Reverse peptides are purported not only to retain the biological activity of the non-reversed “normal” peptide but may possess enhanced properties, including increased biological activity.

Accordingly, a mimetic or mimic of the agent (particularly if a peptide), may be designed for pharmaceutical use. The designing of mimetics to a known pharmaceutically active compound, for example an agent of the invention is a known approach to the development of pharmaceuticals based on a “lead” compound. Mimetic design, synthesis and testing may be used to avoid randomly screening a large number of molecules for a target property. There are several steps commonly taken in the design of a mimetic from a compound having a given target property such as an agent of the invention.

Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”. Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process. In a variant of this approach, the three-dimensional structure of the peptide and its binding partner (haemagglutinin) are modelled. This can be especially useful where the ligand and/or binding partner change conformation on binding, allowing the model to take account of this the design of the mimetic. A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property (binding of haemagglutinin/ability to inhibit viral entry into a cell), or to what extent they exhibit this target property. Further optimisation or modification can then be carried out to arrive at one or more final mimetics for in vivo or clinical testing. Mimetics of an agent identified as having ability to modulate viral activity using a screening method as disclosed herein are included within the scope of the present invention.

An agent or peptide of the invention can be conjugated to various moieties, such as polymeric moieties, to modify the physiochemical properties of the peptide drugs, for example, to increase resistance to acidic and enzymatic degradation and to enhance penetration of such drugs across mucosal membranes. Alternatively, or additionally, the peptide can be provided as a prodrug. The peptides may be present in drug delivery devices as prodrugs. Free amino, hydroxyl, or carboxylic acid groups of the peptides can be used to convert the peptides into prodrugs. Prodrugs can include compounds wherein an amino acid residue, or a polypeptide chain of two or more (e.g., two, three or four) amino acid residues which are covalently joined through peptide bonds are changed to free amino, hydroxy or carboxylic acid groups of various polymers, for example, polyalkylene glycols such as polyethylene glycol. Prodrugs can also include compounds wherein carbonates, carbamates, amides and alkyl esters are covalently bonded to a peptide of the invention through the C-terminal carboxylic acids. Prodrugs comprising a peptide of the invention or pro-drugs from which peptide of the invention (including analogues and fragments) are released or are releasable are considered to be variants of the invention.

Further, a variant can include chimeric or fusion proteins comprising an agent of the invention linked or bonded to another protein, for example an antibody or antibody fragment or defensin. Recombinant fusion proteins can be created artificially by recombinant DNA technology. In embodiments, an agent of the invention can be linked or bonded to an effector molecule wherein the effector molecule can be a small molecule, pharmaceutical drug, toxin, fatty acid, detectable marker or enzyme which can act upon the virus or target cell of the virus to cause an effect. For example the effector may be a cytotoxic small molecule, radioactive isotope, fluorochrome, anti-viral drug or the like.

Compositions

Agents, for example peptides of and for use in the present invention may be administered alone but will preferably be administered as a pharmaceutical composition, which will generally comprise a suitable pharmaceutical excipient, diluent or carrier selected depending on the intended route of administration. Compositions including a peptide of the present invention can be formulated as would be known in the art, for example, a suitable formulation may contain a pharmaceutically acceptable salt form of an agent in particular of the amino acids of a peptide of the invention. Pharmaceutically acceptable salts include both acid and base additional salts and refers to those salts which retain the biological effectiveness and properties of the free bases and which are not biologically or otherwise undesirable.

Compounds and compositions useful in the invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the invention. In general, the compositions of the invention will be formulated such that an effective amount of the bioactive peptide or peptidomimetic is combined with a suitable carrier in order to facilitate effective administration of the composition. For example, conventional pharmaceutically acceptable carriers and diluents which are known to those skilled in the art. Examples of carriers or diluents for use with the subject peptidomimetics include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of an agent of the invention, for example a peptide or peptidomimetic of the invention can also comprise suspension agents, protectants, lubricants, buffers, preservatives, and stabilizers.

An agent of the invention can also be administered utilizing liposome technology, slow release capsules, implantable pumps, nanoparticles, microparticles and biodegradable containers. These delivery methods can, advantageously, provide a uniform dosage over an extended period of time.

The compositions used in the present methods can also be in a variety of forms. These include, for example, solid, semi-solid, and liquid dosage forms, such as tablets, pills, powders, liquid solutions or suspension, suppositories, injectable and infusible solutions, and sprays.

An agent of the invention, for example a peptide can also be modified by the addition of chemical groups, such as PEG (polyethylene glycol). PEGylated peptides typically generate less of an immunogenic response and exhibit extended half-lives in vivo in comparison to peptides that are not PEGylated when administered in vivo. Methods for PEGylating proteins and peptides known in the art (see, for example, U.S. Pat. No. 4,179,337). The subject peptides may also be modified to improve cell membrane permeability. In one embodiment, cell membrane permeability can be improved by attaching a lipophilic moiety, such as a steroid, to the peptide or antibody. Other groups known in the art can be linked to peptides and antibodies of the present invention.

As understood by those in the art, agents, for example peptides with amino acid sequences of the present invention may be provided as esters which are optionally hydrolysable in vivo or in vitro under acidic (pH<three) or basic (pH>than 10). Alternatively, an agent for example a peptide with an amino acid sequences disclosed herein may be substantially stable in the gastro intestinal track of humans, but hydrolysable in the blood or intracellular environment. Alternatively, the amino acid sequences of the invention can be provided in an intermediate form for the preparation of the agent containing the free amino or carboxyl groups. Where an amino acid residue contains one or more chiral centres, any of the D, L, meso or theo or erythro racemates, sclaemates or mixtures thereof may be used. Suitably, enzymes for cleaving amino acid conjugates of the amino acid sequences of the invention may include carboxypeptidases or the like.

Suitably, an agent of the invention, for example a peptide with an amino acid sequence of the invention may be provided with additional amino acids which will provide the amino acid sequences of the invention with transport properties and/or susceptibility to kinases that can affect transport to a cell type. Alternatively, the peptides of the present invention may be provided with additional amino acids to enhance the bioavailability, solubility or solubility of the amino acids sequences of the invention. The addition of amino acid sequences to the peptides of the invention may be selected to provide the peptides of the invention with relative resistance to hydrolysis by proteases found in the body, for example in the lumen of the intestine or the like. Suitably, formulations may be prepared in a sterile form.

Administration of the peptides or peptidomimetics of the invention or polynucleotides encoding the peptides can be continuous or at distinct intervals as can be determined by a person skilled in the art.

The peptides may be administered to a patient in need of treatment via any suitable route. Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. In some embodiments, the peptides can be administered in a suitable capsule or tablet with an enteric coating, so that the peptide is not released in the stomach. In alternative embodiments administration may be by aerosol for pulmonary delivery. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. The composition may also be administered via microspheres, liposomes, other microparticulate delivery systems or sustained release formulations placed in certain tissues including blood.

A unit dosage form can be for example tablet, capsule, lozenge, and powder. Suitably, a tablet may be provided by compression or moulding. Compressed tablets may be prepared by compressing in a suitable machine a relative agent in a free flowing form such as a powder or granules, optionally mixed with a binder, lubricant, inert diluent, preservative, surface active or dispersing agent. Moulded tablets may be made by moulding in a suitable machine a mixture of the powdered active agent moistened with an inert liquid diluents. Optionally, tablets may be coated or scored or formulated to provide a slow or controlled release of the active agent therefrom. In particular embodiments, the active agent may be provided as a lozenge or administration in the mouth. Suitably an agent may be provided with sucrose, gelatine or glycerine. In embodiments an agent may be provided as a mouthwash or for nasal administration or the like. Advantageously, an agent may be provided in an aerosol spray for intranasal delivery or delivery to the lung where it may be suitably located to interact with virus at an entry point into a subject.

The effective dose of the active agent depends at least on the nature of the conditions being treated, and whether the agent is being used prophylactically or against active viral infections, for example influenza infections, the method of delivery and the pharmaceutical formulation.

Methods of Screening for Compounds

Suitably agents of the invention can be used in screening methods to detect further compounds which show inhibitory activity against haemagglutinin using any conventional techniques for the evaluation of an agent binding to a target peptide or polypeptide. Within the context of the invention, typically such further compounds are screened for binding to haemagglutinin and inhibition of virus entry into a cell in vitro and any compounds showing inhibitory activity are then screened for activity in vivo. In particular embodiments, such further compounds are screened for binding to haemagglutinin on influenza and for inhibition of influenza entry into a cell.

According to a further aspect of the present invention there is provided a method to determine an agent which includes a peptide of formula A or a peptide or non-peptide based on an amino acid sequence X₁-X₂-X₃-X₄-X₅-X₆, (SEQ ID NO 1) wherein

X₁ can be phenylalanine, isoleucine or tryptophan;
X₂ can be leucine or phenylalanine or alanine;
X₃ can be tyrosine or valine;
X₄ can be leucine, phenylalanine or isoleucine;
X₅ can be phenyalanine or alanine; and
X₆ can be valine, arginine or tyrosine, capable of binding to haemagglutinin to inhibit entry of a virus, in particular an influenza virus into a cell comprising the steps
a) exposing test cells to a virus, for example influenza virus in the presence and absence of the agent to be tested under conditions which would typically allow the virus, for example influenza, to infect such test cells
b) comparing the number of infected test cells and/or rate of infection of the test cells following exposure to the virus, and
c) determining whether the agent to be tested provides a protective effect and inhibits infection of a cell.

In a first embodiment the method can include a plaque reduction assay. In such an assay, wherein the agent to be tested binds to haemagglutinin, it can inhibit the inhibiting the binding to and infection of cells by the virus, for example by influenza virus. A positive reaction is the reduction of plaques (areas of virus growth) on a susceptible monolayer of MDCK cells. In a second embodiment, the method can include an ELISA used to determine the interaction of peptide with virus using peptide coated plastic surfaces. For example, biotin labelled peptide can be added to strep avidin coated ELISA plate and used as a screen to detect viral haemagglutinin from different virus, for example subtypes of influenza A viruses. Similarly, immobilisation of peptide on suitable plastic or similar material e.g. silicon chips can be used tests such as plasmon surface resonance.

Advantageously, an agent of the invention may be provided in a solution which can be applied to surfaces, for example hard surfaces such as work areas, door handles, equipment such as surgical equipment or where it can be applied to material such that said surfaces or material are conferred with an anti-viral, particularly anti-influenza activity. This provides a further aspect of the invention of an article comprising an agent of the invention coated or impregnated therein. For example, an agent of the invention may be provided to a facemask such that the facemask material can bind to and neutralise virus.

Preferred features and embodiments of each aspect of the invention are as for each of the other aspects mutatis mutandis unless context demands otherwise.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, the singular forms “a”, “an” and “the” include plural reference unless the context clearly dictates otherwise.

As used herein “treating” a condition or subject refers to taking steps to obtain a beneficial or desired result, including clinical results. Beneficial results can include alleviation or amelioration of one or more symptoms of the virus being treated. This can include prophylactic treatment wherein provision of an effective amount of an agent of the invention prevents infection or delays onset of infection by a virus.

As used herein, “subject” refers to an animal or mammal including, but not limited to human, dog, cat, horse, cow, pig, sheep, goat, chicken, monkey, rabbit, mouse, poultry, etc.

As used herein the term “administering” includes both direct administration, including self administration and indirect administration.

Embodiments of the present invention will now be described, by way of example only, with reference to the accompanying figures in which,

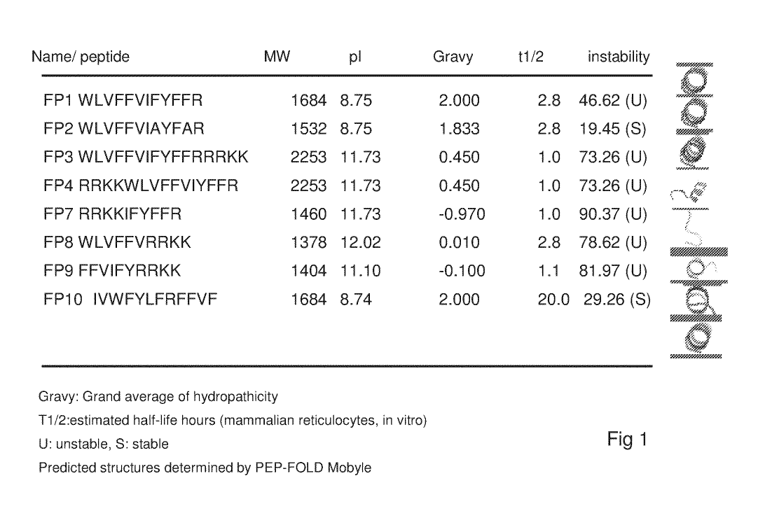

FIG. 1 is a table of amino acid sequences, their chemical and physical properties predicted with the ProtParam algorithm (MW=molecular weight, pl=isoelectric point, GRAVY=Grand average of hydropathicity, t½: estimated half-life hours (mammalian reticulocytes, in vitro), instability: U=unstable, S=stable), and predicted peptide secondary structures determined by PEP-FOLD Mobyle,

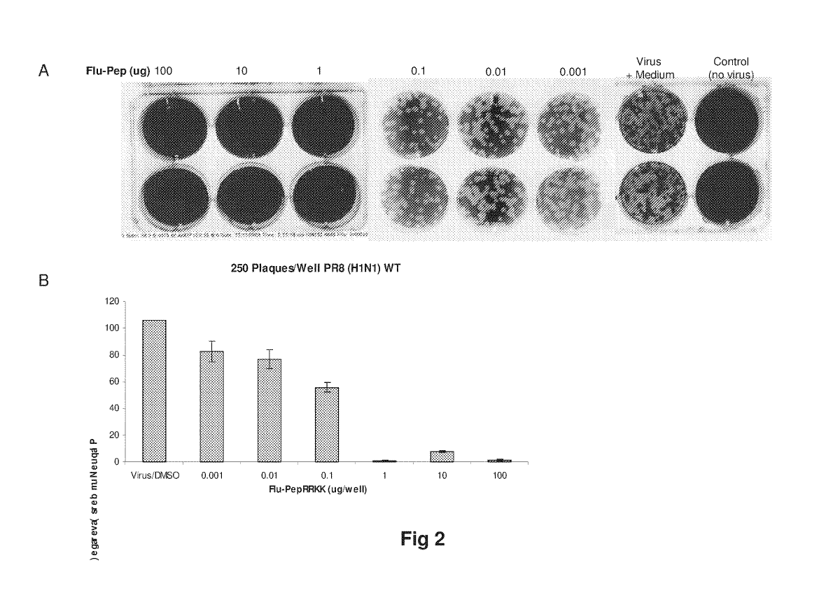

FIG. 2 is A) a photograph of a plaque reduction assay test plate for determining an antiviral effect of a FP1 derived peptide (FP3) by determining inhibition of PR8 (H1N1) virus infection of MDCK cells, wherein PR8 (H1N1) is diluted at 250 PFU per test well, by double testing (top row, bottom row), wherein (from left to right) 100, 10, 1, 0.1, 0.01, 0.001 μg FP3 peptide was added to the respective test well, followed by a positive control (medium with cells and virus) and negative control (medium with cells, no virus), and B) a bar diagram showing a plaque number (average) at different FP3—RRKK peptide masses per test well containing MDCK cells and PR8 (H1N1) diluted at 250 PFU per well for, from left to right) a DMSO control (virus and DMSO) and 0.001, 0.01, 0.1, 1, 10, 100 μg FP3—RRKK per well, standard deviation is indicated at the top of each bar where applicable,

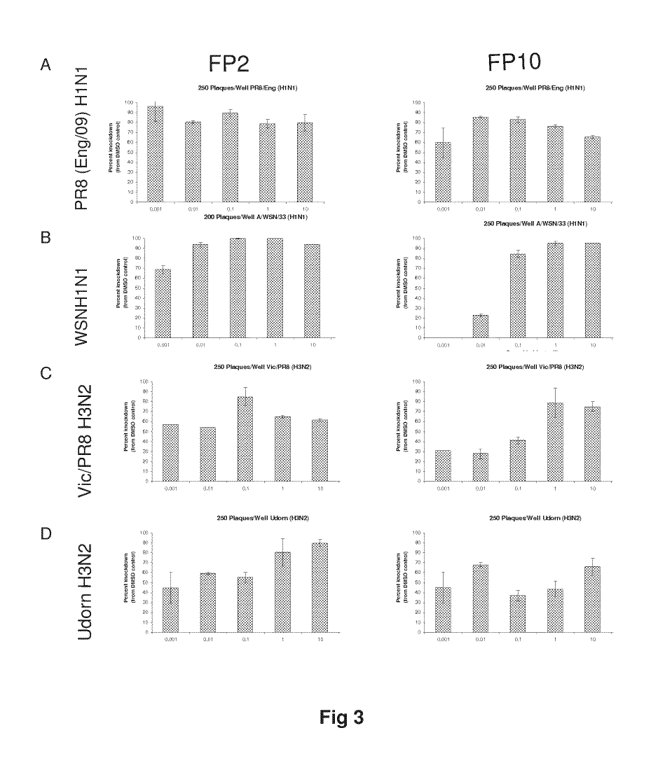

FIG. 3 is a compilation of bar diagrams showing plaque reduction, in % compared to a DMSO control, at no (DMSO control with virus and DMSO), 0.001, 0.01, 0.1, 1, 10 μg FP2 peptide (left side) and FP10 peptide (right side) per test well containing MDCK cells, wherein the standard deviation is indicated at the top of each bar where applicable, and in which A) PR8 (Eng/09) H1N1 virus was diluted at 250 pfu per well tested (left and right side), B) A/WSN/33 H1N1 virus was diluted at 200 pfu per well tested (left side) and 250 pfu per well tested (right side), C) Vic/PR8H3N2 virus was diluted at 250 pfu per well tested (left and right side), and D) Udorn H3N2 virus was diluted at 250 pfu per well tested (left and right side), Four replicates/sample and Results are a representative of 2 to 4 experiments,

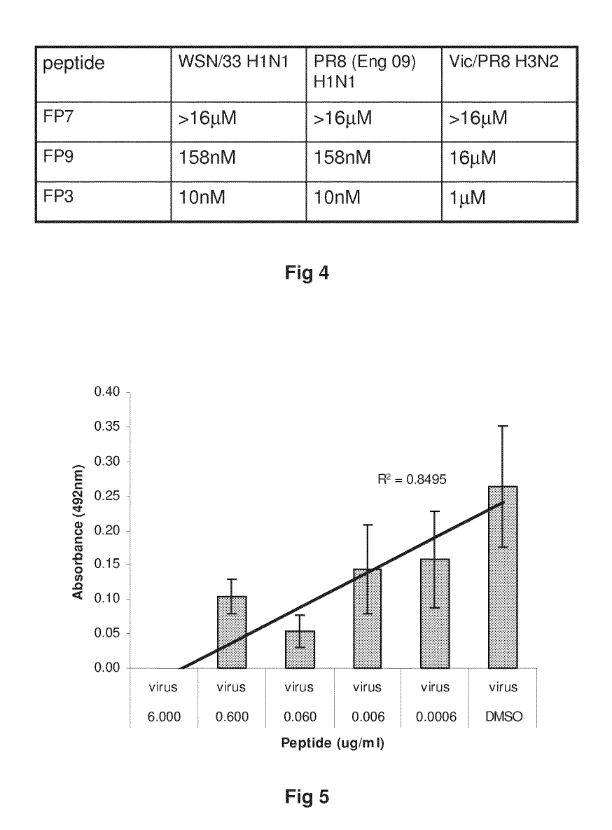

FIG. 4 is a table showing the IC₅₀ of truncated FP peptides (top row to bottom row: FP7, FP9 and FP3 against A/WSN/33 H1N1, A/Engand/09/PR8H1N1 and A/Vic/PR8H3N2 virus (second to fourth column), wherein FP7 and FP9 were compared against each other and FP3 in a plaque reduction assay in which FP7 (RRKK-IFYFFR) failed to inhibit infection with H1N1 and H3N2 virus and FP9 (FFVIFYRRKK) was very effective at inhibiting H1N1 infection, but was not as effective as FP3 (WLVFFVIFYFFRRRKK),

FIG. 5 illustrates the results of an influenza adsorption assay which was used to determine if peptides of the invention inhibit adsorption of Influenza virus onto cell surfaces in a bar diagram, in which inhibition of binding of WSN (moi 6) virus to MDCK cells was determined by measuring absorbance at 492 nm of test reactions with 6, 0.6, 0.06, 0.006, 0.0006 μg FP3 peptide per mL virus suspension, wherein chilled MDCK cells were exposed to WSN virus suspension with FP3 peptide (diluted in DMSO at a final concentration of 1.5% v/v) for 1 hour at 4° C., fixed and probed for attached virus using polyclonal anti-Influenza A (strain USSR H1N1) and an HRP-conjugated secondary antibody, and wherein the plates were developed using the SigmaFAST OPD kit and read at 492 nm and the reaction was stopped by addition of stop solution; mean values and standard errors of 4 experiments are provided,

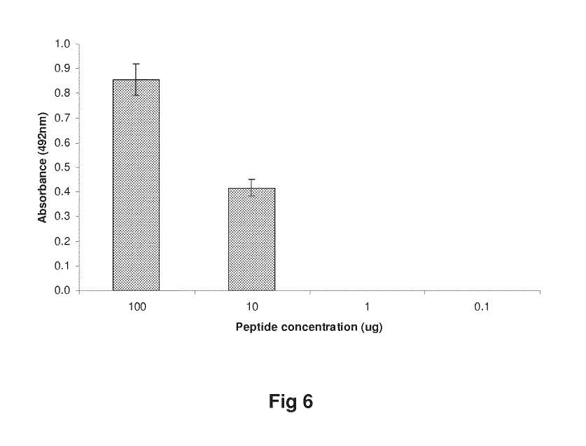

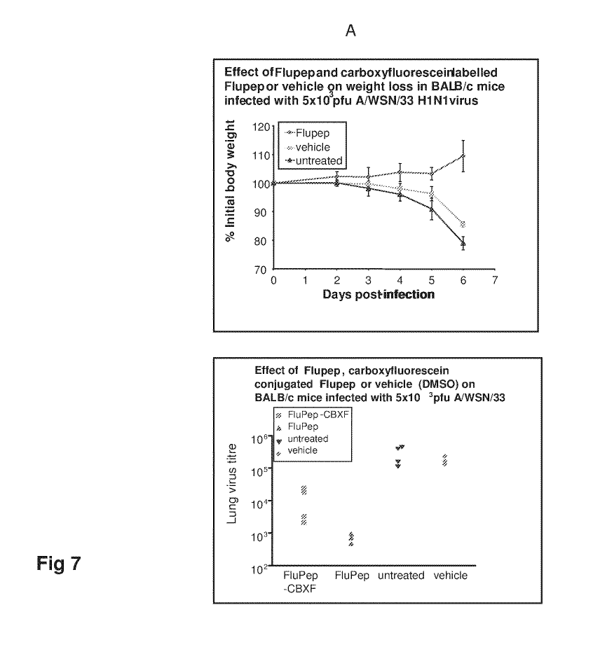

FIG. 6 illustrates the result of an influenza haemagglutinin binding assay (ELISA) using a bar diagram, in which absorbance at 492 nm was determined for Flu-peptide FP3 concentrations of 100, 10, 1 and 0.1 μg/well, wherein microtiter plates were coated with increasing concentrations of Flu-peptide FP3, washed, blocked with BSA, incubated with 0.01 ug/well baculovirus-derived recombinant Haemagglutinin (HA) (California 04/2009 H1N1 Influenza A), and which was detected with anti-Influenza A (strain USSR H1N1) antibody and an HRP-conjugated secondary antibody; the plates were developed using the SigmaFAST OPD kit and the reaction was stopped by the addition of stop solution; absorbance was determined at 492 nm after; mean values and standard errors of 3 experiments are provided, An interaction between peptide and HA provides an explaination of the inhibition of virus binding to the surface of MDCK cells. The results of this ELISA clearly show that peptide can bind Influenza A HA,

FIG. 7 shows diagrams indicating the effect of Flupep FP1 and carboxyfluorescein labeled Flupep FP1 and of the vehicle (DMSO) on BALB/c mice infected with 5×10³ pfu A/WSN/33 H1N1 virus and of untreated BALB/c mice, wherein (top diagram) the effect was determined as % initial body weight at 0 to 7 days (daily determination) post infection for Flupep and vehicle (DMSO) treated and untreated mice (standard deviation is indicated for the respective value point, where applicable), and (bottom diagram) the effect was determined as lung virus titre at 7 to 8 days for Flupep, carboxyfluorescein labeled Flupep (FP1—CBXF) and vehicle (DMSO) treated and untreated mice,

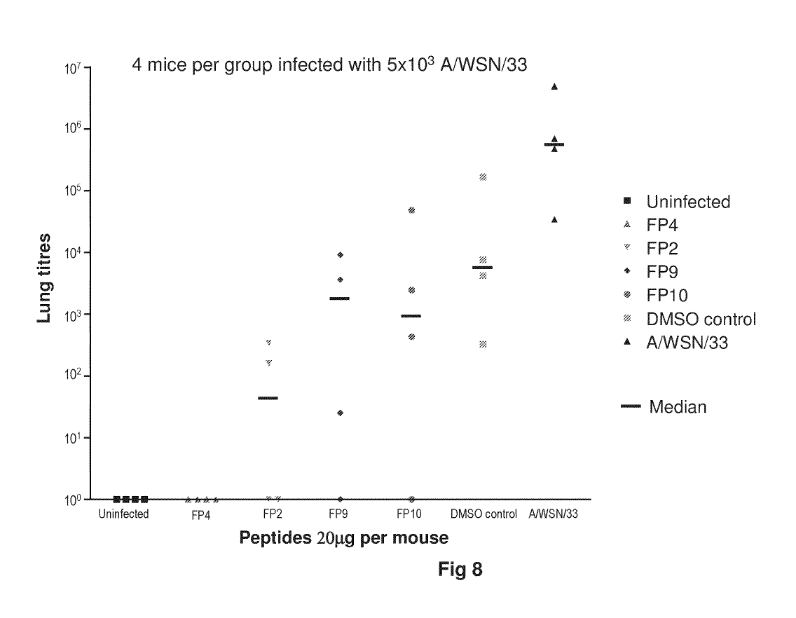

FIG. 8 shows a scatter diagram of a virus lung titre of BALB/c mice infected with 5×10³ pfu A/WSN/33 virus, each, (4 mice infected per group) and inoculated with 20 μg peptide (FP4, FP2, FP9, FP10) in 40 μl 2% DMSO in PBS, uninfected mice, mice inoculated with DMSO (DMSO control) and untreated virus infected mice, wherein the virus yield in the lung was determined on day 7 post infection; the median values are indicated by a bar,

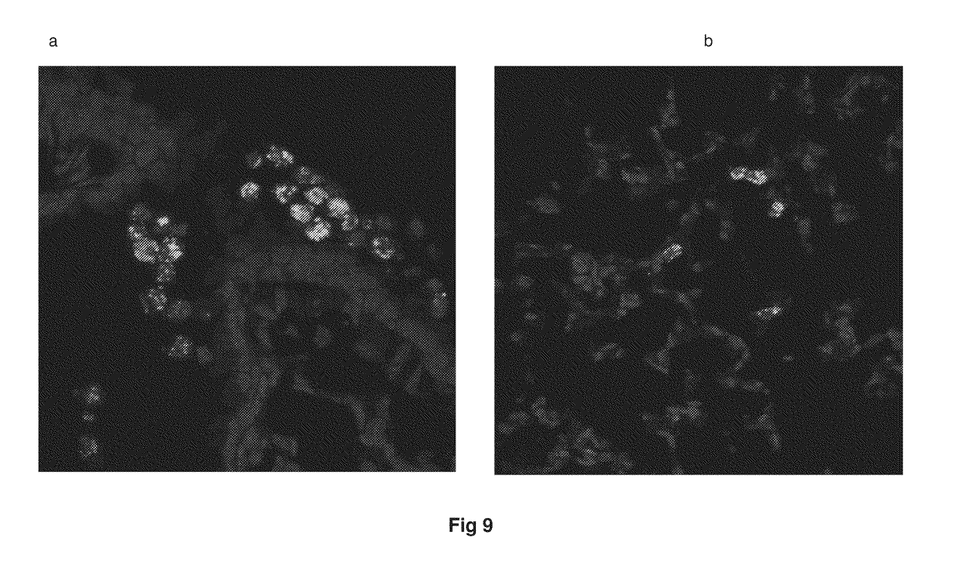

FIG. 9 A shows a peptide of the invention in uninfected mouse lung cells 18 days after administration B shows peptide of the invention in A/WSN/33 infected Balb/c mouse lung cells 7 days post infection,

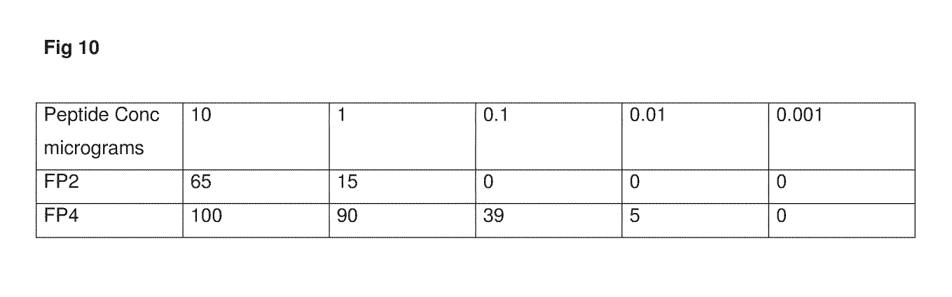

FIG. 10 shows the percentage knock down of A/Vietnam/1194/04PR8 (H5N1) plaque formation following treatment with varying doses of either FP2 or FP4,

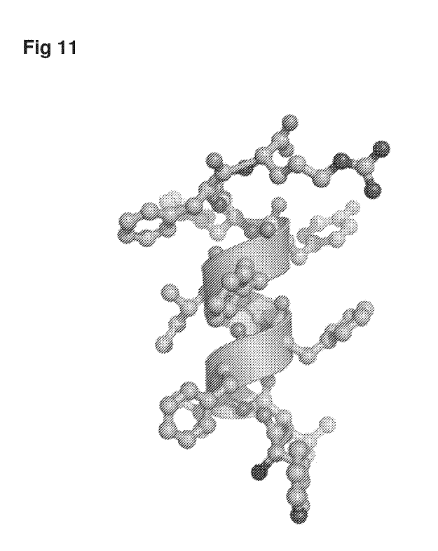

FIG. 11 shows a predicted three dimensional structure of a peptide of the invention,

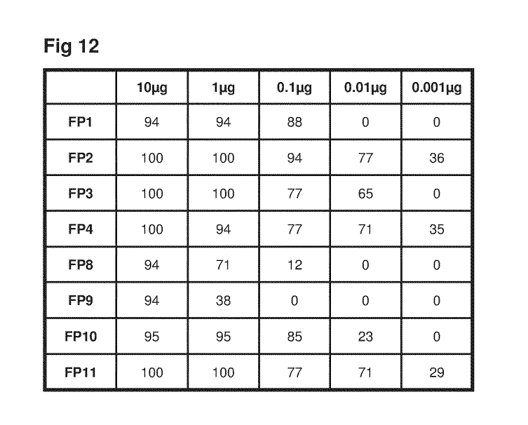

FIG. 12 illustrates the percent knockdown of A/WSN/33 (H1N1) by peptides in MDCK plaque reduction assays whereby modifications to the peptide FP1 WLVFFVIFYFFR (SEQ ID NO 18) resulted in improved antiviral activity against H1N1 virus. Truncated peptides (6-10 mers, see FP8 and FP9) still maintain substantial anti-viral activity,

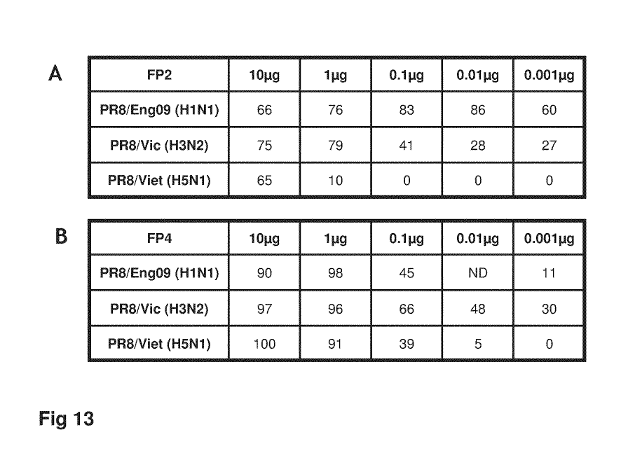

FIG. 13 illustrates the percent knockdown of various subtypes by peptide FP2 (FIG. 13A) and FP4 (FIG. 13B) in MDCK plaque reduction assays using subtypes including A/England/195/09 (H1N1), A/Victoria/3/75 (H3N2) and A/Vietnam/1194/04 (H5N1) recombinant viruses containing PR8 internal genes with haemagglutinin (HA) and neuraminidase (NA) subtypes listed,

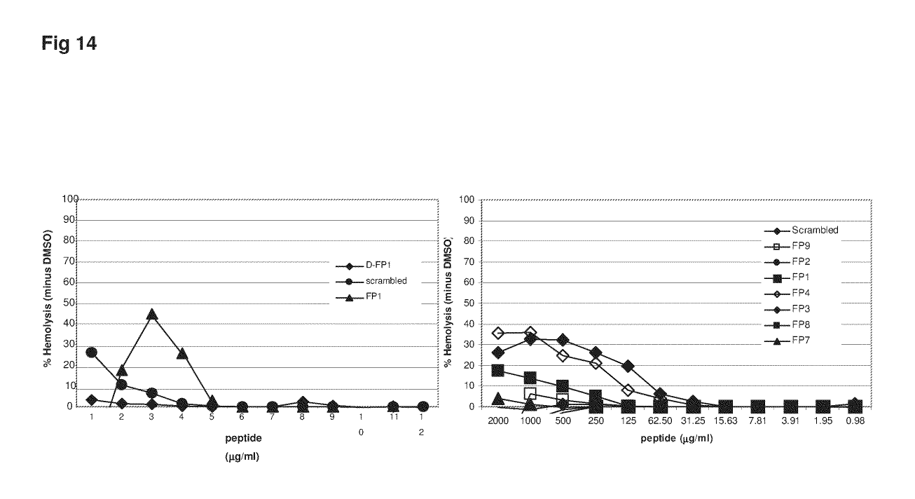

FIG. 14 illustrates the results of a hemolysis assay to consider the toxicity of peptides of the invention whereby the assay measures the release of hemoglobin from non-viable cells which is quantified using a spectrophotometer. Human Type 0 red blood cells were mixed with various amounts of peptides for 4 hours (final concentration e.g. 2000-0.98 μg/ml), 37° C. and 5% CO2. The hemoglobin released into the supernatant was measured at wavelength 540 nm. The percent hemolysis was calculated using the equation of the straight line, where % hemolysis (x)=[optical density (y)−negative control optical density (c)]/[(positive control optical density−negative control optical density)/100](m). Peptides were provided at 2.5 μg/ml=1 ug per well in the plaque assays. 0.0025 μg/ml which is well below the lowest toxic concentration,

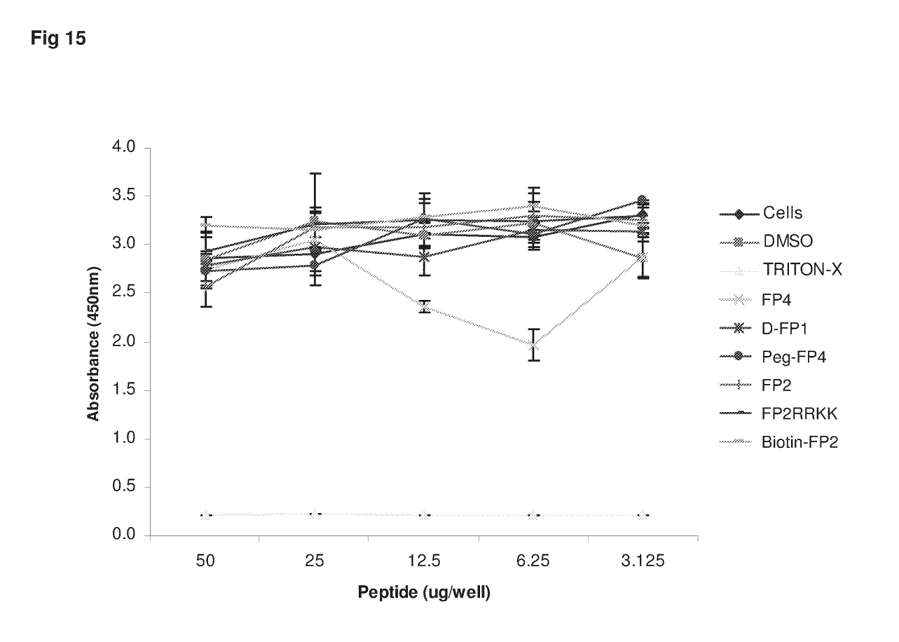

FIG. 15 shows the results of a further assay to test the toxicity of the peptides whereby the assay measures cell viability; cellular enzymes in viable cells cleave the tetrazolium salts in WST-1 to the dye formazan, which is quantified using a spectrophotometer. Culture cells in microplates in 100 μl culture medium containing various amounts of peptides for 24 hours (final concentration e.g. 3.125-50 μg/well), 37° C. and 5% CO2. Cell proliferation reagent WST-1 is incubated with cells for 2 hours and absorbance of the formazan product is read at 450 nm,

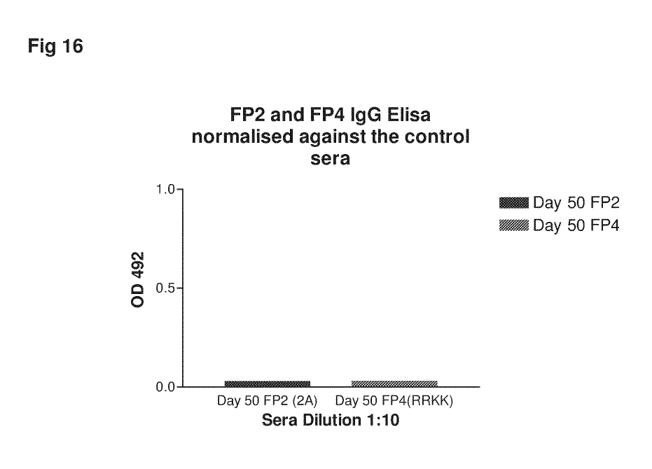

FIG. 16 shows the results of assays to determine the immunogenicity of peptides FP2 and FP4 whereby 20 μg of peptide in 2.5% DMSO was administered through intranasal route in a 40 μl volume to 6 week old, female Balb/c mice. Serum was obtained 1 week prior to and days 7, 14, 28 and 50 post-day 0. Peptide was administered on days 0, 10, and 21,

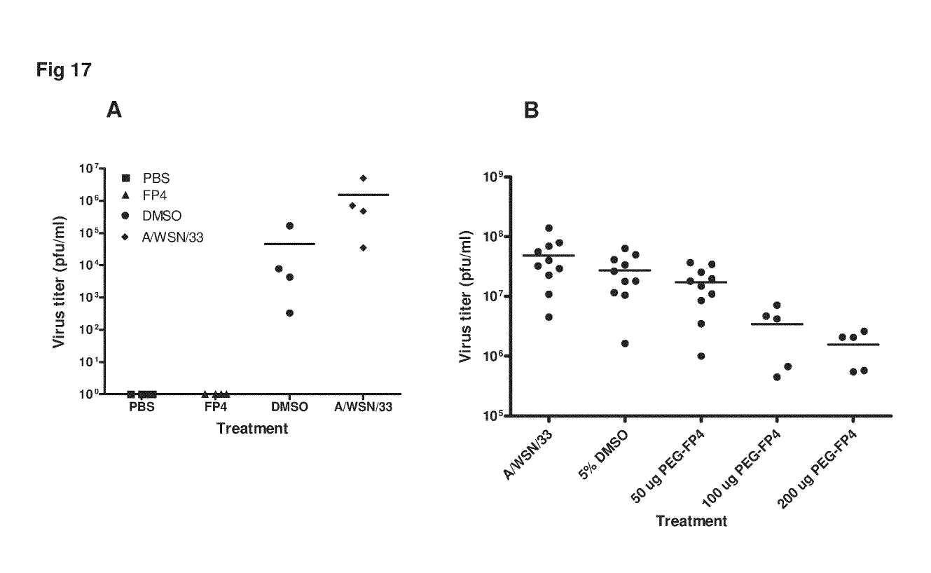

FIG. 17 shows the results of an assay determine the antiviral effects of FP4 peptide (A) or pegylated FP4 peptide (B) whereby 2.5-5% DMSO was administered through intranasal route in a 50 μl volume to 6 week old, female Balb/c mice. Peptide was administered at the same time as virus (A) or 24 and 48 hours after virus (B),

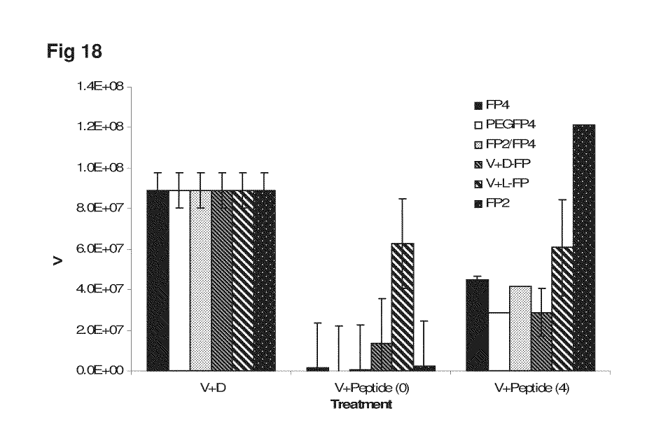

FIG. 18 shows that if peptides of the invention are provided at the same time as virus, in vitro or in vivo, this dramatically limits virus replication/infection,

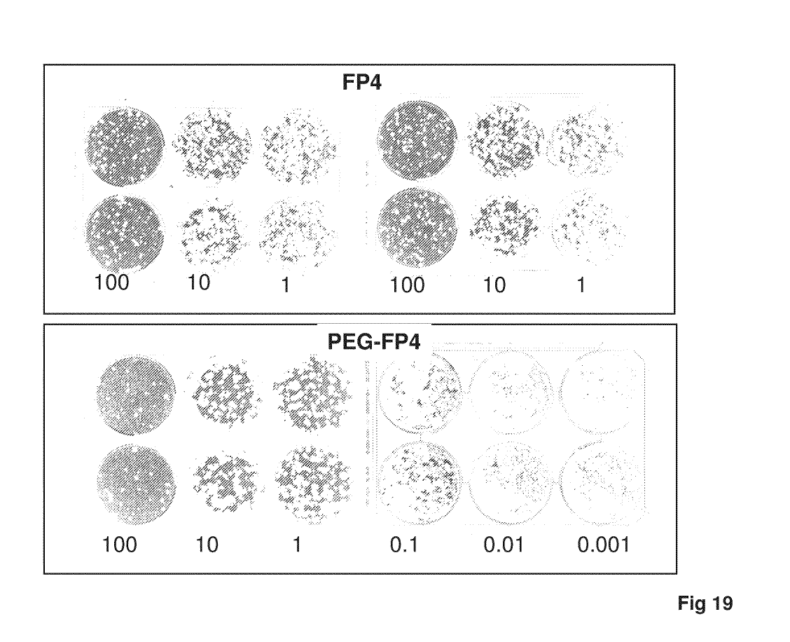

FIG. 19 illustrates the results of a parainfluenza plaque assay wherein virus at 100 pfu per well was incubated for 1 h with the indicated amount of peptide and Infected cell monolayers are overlaid with Avicel (carboxymethyl cellulose) and left for 10 days.

MATERIALS AND METHODS

Mice And Virus Infections

BALB/c mice were purchased from Harlan UK Ltd (Oxon, UK). All work was carried out under a UK Home Office license according to the Animals (Scientific Procedures) Act 1986. Five- to 6-week-old female mice were used in all experiments. Virus working stocks were prepared by infection of Madin-Darby canine kidney (MDCK) cells and titrated on MDCK cells by standard plaque assays. Mice were anesthetized using Halothane or isofluorane (Rhone Merieux Ltd, Harlow, Essex, UK) and infected intranasally with either 5×103 PFU of A/WSN/33 influenza virus in 40 μl PBS, or 100 PFU in 50 μl volume, in the presence or absence of peptide. For therapeutic evaluation of the peptide mice were subsequently anesthetized 24 and 48 hours post-viral inoculation. Mice were weighed daily and assessed for visual signs of clinical disease, including inactivity, ruffled fur, and laboured breathing. Animals that had lost >25% of their original body weight were euthanized. At various times after infection, mice were euthanized by CO2 asphyxiation, and the lungs removed, homogenized in PBS and clarified by centrifugation. Titers of infectious virus were determined by standard plaque assays on MDCK cells.

To determine the immunogenic potential of these peptides we administered 20 μg/mouse of FP2 or FP4 peptide to 5-6-week-old female BABL/c mice on three separate occasions; day 0, 10 and 21. Blood was obtained 1 week prior to first administration of peptide and on days 7, 14, 28 and 50 after the first peptide dose. Serum was used for the analysis of IgG by ELISA. Briefly, microtiter plates were coated with 10 ng of FP2 or FP4 peptide for 24 hours at room temperature, followed by addition of serum (1:10-1:1000) and detection of mouse IgG using goat anti-mouse IgG HRP antibody (Abd Serotec, UK) followed by SIGMAFast™ OPD (Sigma Aldrich, St Louis, Mo.). Plates were read at 492 nm after addition of 3M H2SO4 stop solution.

Viruses

Influenza viruses as discussed herein are: A/WSN/33 H1N1, A/PR8/34 H1N1, A/England/195/09/PR8H1N1, A/Victoria/3/75/PR8 (H3N2), A/Udorn/72 H3N2 and A/Vietnam/1194/04/PR8 (H5N1). Main Darby canine kidney epithelial cells (MDCK) and human lung epithelial cell line A549 (ATCC), were maintained in Dulbecco's modified Eagle's medium (DMEM) (Nitrogen, GIBCO) supplemented with 10% fatal bovine serum (FBS) (Invitrogen, GIBCO), 50 U/ml penicillin and 50 μg/mlstreptomycin (Invitrogen, GIBCO). All viruses were propagated in MDCK cells prior to use in the study. The A/WSN/33 (H1N1) and Udorn (H3N2) influenza viruses were obtained from Dr D Jackson, St Andrews. All other viruses were kindly provided by Professor Wendy Barclay, Imperial College, London. Recombinant variants of A/Puerto Rico/3/34 (PR8; NIBSC strain) were obtained using a reverse genetics system, as previously described (Whiteley A; Major D; Legastelois I; Campitelli L; Donatelli I; Thompson C I; Zambon M C; Wood J M; et al. (July 2007). Generation of candidate human influenza vaccine strains in cell culture—rehearsing the European response to an H7N1 pandemic threat. Influenza

Other Respi Viruses. 1:157-166.); A/England/195/09 (H1N1), A/Victoria/3/75 (H3N2), A/Vietnam/1194/04 (H5N1), A/Chicken/Italy/13474/99 (H7N1). Virus titers were obtained by performing standard plaque assays on MDCK cells. Briefly, serial dilutions of virus were added to confluent monolayers of cells for 1 hour, 37° C. with 5% CO2. Following the 1 hour incubation, unincorporated virus was removed and monolayers overlaid with 1% agarose and incubated at 37° C., 5% CO2 for three days, after which time the cell monolayers were fixed with 10% neutral buffered formalin, stained with 0/1% Toluene blue, and plaque numbers obtained.

Peptides

Peptides were manufactured and purified by high-pressure liquid chromatography (Cambridge Peptides, Cambridge, UK). Additional peptides were synthesized within the School of Chemistry, University of Edinburgh. The peptide panel included peptides containing either the amino- or carboxyl-RRKK sequence to increase solubility. FP2 has two alanine substitutions. Peptides FP7, 8, and 9 are truncated peptides, and FP10 is a scrambled sequence of peptide FP1. In addition, to enhance bioavailability and protect against proteolytic degradation FP1-D was prepared with D-amino acids (SEQ ID NO 20), and FP4 was pegylated by linking the peptide with polyethylene glycol chains to provide PEG300-RRKKWLVFFVIFYFFR (SEQ ID NO 21). These synthetic polymers (PEG) are approved by FDA for internal use, and are known to be non-immunogenic and non-antigenic in nature.

Influenza Virus Plaque Assay

6 well plates with 2×10⁶ MDCK cells per well set up two days before assay and serial dilutions of virus were prepared from 10⁻² to 10⁻⁸. The medium was removed from the 6 well plates and wells were washed twice with sterile PBS.

400 μl of the 10⁻² to 10⁻⁸ dilutions was added to duplicate wells. The plates were incubated at 37° C. and rocked every 10 minutes for 1 hr. 2% agarose (Biogene.com cat no 300-200) in sterile distilled water was melted and maintained at 55° C. 10 μl of a 10 mg/ml stock of NAT (N-acetyl trypsin bovine pancreas type V-S Sigma T6763) was added to 50 ml of 2× serum free DMEM to give a final concentration of 2 μg/ml and medium was maintained at 37° C. After 1 hr the virus dilutions were removed from the plates. 25 ml of the 2% agarose was quickly added to the 25 ml aliquots of media, mixed well and 2 ml added to each well. After setting the plates were inverted and incubated at 37° C. for 3 days. Plates were fixed with 4 ml of 10% neutral buffered formalin (Surgipath Europe 00600) overnight, the agarose was removed leaving the adherent cells which were stained with 0.1% toluidene blue 0 (Sigma T3260) for 20 minutes. Plates were washed with water and plaques were counted. Virus stocks were stored at −70° C.

### Plaque Reduction Assay

MDCK cells were plated at 2×10⁶ cells per well in 6 well plates as for the viral plaque assay. Confluent monolayers of MDCK cells (ATCC) were grown in 6 well dishes and infected with a dilution of virus required to obtain 250 plaques per well. Virus adsorption was carried out in the presence or absence of peptide for 1 hour, 37° C. with 5% CO2, in a total volume of 400 μl. For plaque reduction assays unincorporated virus and/or peptide was removed with the addition of an overlay containing complete DMEM, 1% agarose and 2 μg/ml trypsin (NAT, acetylated from bovine pancreas)(Sigma Aldrich, St Louis, Mo.), final concentrations. The incubation was continued for 72 hours, 37° C. with 5% CO2, followed by fixation with neutral buffered formalin, and staining of the cell monolayer with 0.1% Toluidine blue (Sigma Aldrich, St Louis, Mo.), and virus concentrations as determined by plaque numbers, plaque-forming units, was obtained. For viral growth assays, after the initial 1 hour incubation with virus and/or peptide the 400 μl inoculums were increased to a volume of 2 ml with complete DMEM medium containing NAT (2 μg/ml). Supernatants were collected at 24-72 hours, and viral titers determined by plaque reductions assays (detailed above).

Assuming 2×10⁶ cells per plate, the virus was diluted to give a moi of 0.001 or 250 PFU per well in a volume of 400 μl/well. Peptides, dissolved in DMSO, were added to virus at concentrations of 100 ug to 0.001 ug/well in a final concentration of 1.1% DMSO. Plates were processed as for plaque assays. Controls contained virus and DMSO only.

Virus Adsorption ELISA

Mouse adapted A/WSN/33 (H1N1) virus was incubated with increasing concentrations of peptide (for example 0.0006-6.0 μg/ml) for 1 hour at 37° C. and chilled. The virus or virus and peptide solutions were plated on chilled MDCK cells in 96-well plates (50 μl/well, M.O.I of 6), for 1 hour at 4° C., washed with cold phosphate buffered saline (PBS), fixed with 4% paraformaldehyde at room temperature for 30 minutes, washed with cold PBS and stored overnight at 4° C. Plates were blocked with 3% bovine serum albumen (BSA) in tris-buffered saline (TBS) for 1 hour at room temperature, probed with 1:500 dilution of goat anti-Influenza A antibody (AbD Serotec, UK) for 1 hour at 37° C. Plates were washed with PBS, and probed with 1:500 dilution of HRP conjugated secondary antibody, donkey anti-sheep/goat IgG HRP (AbD Serotec, UK) for 1 hour at 37° C., washed with PBS and developed using 200 μl/well SIGMAFast™ OPD (Sigma Aldrich, St Louis, Mo.) at room temperature for 30 minutes, covered. Plates were read at 492 nm after addition of 3M H2SO4 stop solution.

Influenza Haemagglutinin Binding Assay (ELISA) Dynex Immulon 4HBX flat-bottom microtiter plates were coated with peptide of the invention diluted in phosphate buffered saline containing 10% DMSO. Coating was performed in triplicate at room temperature for 18 hours with 5 μg/well (100 μg/ml). Plates were washed and then blocked with 1% BSA TBS for 1 hour at room temperature. They were then washed with TBST (0.1% Tween 20). Purified baculovirus-derived recombinant HA

(Haemagglutinin) (California 04/2009 H1N1 Influenza A, Source BioScience AUTOGEN #ABC1278) was added at a concentration of 0.01 μg/well (50 μl volume) at room temperature for 2 hours. After extensive washing, peptide bound rHA (recombinant Haemagglutinin) was detected with anti-influenza antibody (goat anti-Influenza A, AbD Serotec) and an HRP-conjugated secondary antibody (donkey anti-sheep/goat IgG-HRP, AbD Serotec). Plates were washed with PBS, and probed with 1:500 dilution of HRP conjugated secondary antibody, donkey anti-sheep/goat IgG HRP (AbD Serotec, UK) for 1 hour at 37° C., washed with PBS and developed using 200 μl/well SIGMAFast™ OPD (Sigma Aldrich, St Louis, Mo.) at room temperature for 30 minutes, covered. Plates were read at 492 nm after addition of 3M H2SO4 stop solution.

Cytotoxicity Assays

Serial dilutions of peptides were made in 0.9% normal saline to which 30% human blood type 0 red blood cells were added and incubated at 37° C. for 4 hours. The amount of haemoglobin released into the supernatant was determined spectrophotometrically at wavelength 540 nm. Negative control wells contained red blood cells in saline alone, and positive control wells were treated with 0.1% Triton-X-100. Percent haemolysis was calculated using the equation of the straight line; y=mx+c, where % hemolysis (x)=[optical density (y)−negative control optical density (c)]/[(positive control optical density−negative control optical density)/100](m). To determine the toxicity of peptides on MDCK cells and human lung epithelial cell line A549, cells were cultured in tissue culture grade 96 well microplates with various concentrations of peptides for 24 hours at 37° C., 5% CO₂. Cell viability was determined by incubation with WST-1 reagent for 2 hours (Roche), measuring absorbance of the formazan product at 450 nm.

Haemagglutination Assay

Influenza virus haemagglutinin (HA) is capable of agglutination of red blood cells (RBCs). This assay was used to investigate whether peptides could be used to block HA binding (haemagglutination inhibition assay). Briefly, virus/peptide mixtures diluted in saline were plated on 96-well round-bottom plates in a volume of 50 μl, and mixed with an equal volume of 1% red blood cells (RBCs) at 4° C. overnight. Non agglutinated cells form a button pellet at the bottom of the well. Agglutinated RBCs coat the well evenly. The titre is determined as the last dilution that shows complete agglutination.

87.5 μl of saline was added to column 1 of 96 well round bottomed plates and 50 μl to all remaining wells. 12.5 μl of virus or peptide was added to appropriate wells in lane 1. Lane 1 was mixed with a multi-channel pipette and 50 μl was transferred to lane 2. Two-fold dilutions were continued to the end of plate. 50 μl/well of red blood cells was added and the plate was incubated on ice at 4° C. overnight.

In general, the starting concentration of peptides in lane 1 was 100 μg/ml. The starting concentration for virus was around 10⁸ pfu/ml. Human red blood cells, Type 0 or Type B, 1% packed cell volume, were washed in saline and stored at 4° C. Other human blood types and species RBCs (sheep and horse) were also tested.

Results

Physical and Chemical Properties of FP1 and Peptides Derived Therefrom And Modifications to Improve Solubility in Aqueous Solution.

Several peptides were developed based on the original structure of the 12 mer referred to as FP1 (FIG. 1). The FP1 12 mer was predicted to have an alpha-helical structure and be hydrophobic. The amino acid sequences and physical and chemical properties of these peptides, as used in the present study were determined by the ExPASy-ProtParam tool and the results of this determination are presented in FIG. 1. The majority of the peptides are hydrophobic and were predicted to exhibit a coiled structure. For use in tissue culture and in experiments in mice the peptides were initially dissolved in DMSO and then diluted in tissue culture medium to give a 2% DMSO solution. Solubility was determined to be improved by the addition of RRKK to the N- or C-termini of the peptide without compromising the anti-viral activity. Further improvements involved replacing arginine with lysines at the N-terminus, or C-terminus or both the N- and C-termini.

Peptide synthesis was carried out at the School of Chemistry, University of Edinburgh or at Cambridge Peptides, Cambridge UK. Purity was checked using HPLC and the sequence specificity was determined using Mass Spec.

Examples are Presented Illustrating the Anti-Influenza Virus Properties of FP1 and its derivatives.

### EXAMPLE 1

### Assessment of Anti-Influenza Activity of Peptide in Plaque Reduction

Using a plaque reduction assay, peptides of the invention were tested to determine their ability to inhibit replication of influenza virus. A representative set of viruses, including mouse adapted A/WSN/33 (H1N1), human PR8 recombinant viruses A/Victoria/3/75 (H3N2), A/England/195/09 (H1N1), A/Vietnam/1194/04 (H5N1), and A/Chicken/Italy/13474/99 (H7N1) were tested. PR8 recombinant viruses have an A/Puerto Rico/8/34 backbone with HA and NA replaced from the designated viruses listed. Assays were carried out with virus in the presence of vehicle (DMSO, 1.5% final concentration), or virus in the presence of increasing concentrations of peptide.

The peptide of the invention FP1, was found to be highly efficient at inhibiting the mouse adapted A/WSN/33 H1N1 subtype, with 94% knockdown of plaque formation with doses of 1-10 μg/well of peptide (FIG. 2). Modifications to this peptide, to form peptides FP2, FP3 and FP4 resulted in improved antiviral activity against the A/WSN/33 H1N1 virus (FIG. 3), resulting in 100% knockdown. Truncated peptides (FP8 and FP9), still maintain substantial antiviral activity against the H1N1 virus, resulting in 94% knockdown, similar to the original peptide, FP1. Therefore this family of peptides is capable of inhibiting the H1N1 mouse adapted influenza virus in vitro.

Peptides of the invention designated FP3 and FP4 (SEQ ID NO 12 and SEQ ID NO 13) (derivatives of FP1 with the addition of RRKK to either the N terminal or C terminal of the FP1 sequence) were found to efficiently inhibit infectivity of H1N1 viruses—A/WSN/33 and A/England/195/09 (IC₅₀ 66-100 nM) (FIG. 4).

The antiviral activity of the peptides against other subtypes of influenza virus was also investigated. Recombinant viruses containing the PR8 internal genes with HA and neuraminidase (NA) from various H3N2, H5N1 and H7N1 were tested in plaque reduction assays in the presence of FP2 or FP4. Treatment with 10 μg of FP4 resulted in 90% knockdown of the PR8/Eng09 H1N1 virus, compared to 100% knockdown of the A/WSN/33 mouse adapted H1N1, as well as 97% knockdown of the PR8/Vic H3N2 virus, and complete knockdown of the PR8/Viet H5N1 virus. Treatment with 10 μg of FP2 peptide, which results in complete knockdown of the A/WSN/33 H1N1 virus, knockdown by 66%, 75% and 65% the PR8/Eng09 H1N1, PR8/Vic H3N2 and PR8/Viet H5N1, respectively. FP1 had no antiviral effects against murine gammaherpes virus 68 (MHV-68), Semliki Forest virus (SFV), or human parainfluenza virus (data not shown). Therefore, these peptides are effective at inhibiting influenza A virus at μM to nM concentrations within in vitro model systems.

FP3 was found to only weakly inhibit the H3N2 viruses A/Udorn and A/Victoria/3/75 (IC₅₀₋16 μM). An example of a plaque reduction assay for FP3 is shown in FIG. 2

In contrast, FP2 (SEQ ID NO 11) was determined to be highly efficient at inhibiting both H1N1 and H3N2 viruses (IC₅₀₋1.6 nM). Another variation on FP1 is FP10 (SEQ ID NO 14). This peptide retains a coiled secondary structure and was determined to be remarkably effective against both H1N1 (IC₅₀<1.48 nM) and H3N2 (IC₅₀₋72 nM) influenza virus with (Examples of inhibition by FP3, FP7, FP9, FP2 and FP10 against different influenza A viruses are shown in FIGS. 3 and 4).

### EXAMPLE 2

### Effect of Truncating Peptides of FP1

Truncated forms of FP1 were generated comprising 6 amino acids representing the NH2 (amino acids 1-6, FP7) and COOH (amino acids 7-12, FP8) termini and a middle section (amino acids 4-9, FP9). RRKK was added to each 6 mer to improve solubility in aqueous solution enabling higher doses of the peptide to be administered in vitro and in vivo. In a plaque reduction assay FP9 was active against H1N1 (IC₅₀₋1.5 μM) and to a lesser extent against H3N2 (FIG. 4). FP7 failed to inhibit influenza virus infection.

### EXAMPLE 3

### Efficacy of a D-Isomeric Form of FP1-Derived Peptide (WLVFFVIFYFFR-SEQ ID NO 18)

An antimer (D-isomer) of FP1 was constructed that inhibited both H1N1 and H3N2 viruses in vitro. Peptides containing D-isomers are more resistant to protease digestion and therefore able to persist in the host for significantly longer than the L form. The D isomer of FP1 was shown to inhibit H1N1 and H3N2 virus infection at 100 nM.

### EXAMPLE 4

### FP1 and Peptides Derived Therefrom Inhibit Binding of Virus to Cells

To investigate the role of FP1 and peptides derived therefrom as an entry blocker chilled MDCK cells were exposed to WSN (m.o.i 6) and increasing concentrations of peptide (diluted in DMSO) for 1 hour at 4C, fixed and probed for attached virus using polyclonal anti-Influenza A (strain USSR H1N1) and an HRP-conjugated secondary. The results shown in FIG. 5 indicate that FP1 and peptides derived therefrom act as entry blocker.

### EXAMPLE 5

### FP1 and Peptides Derived Therefrom Bind to Haemagglutinin

To determine the mechanism for protection of mice examination of whether peptides of the invention inhibited attachment of influenza virus onto MDCK cell surface was undertaken. Chilled tissue-culture treated microtiter plates coated with MDCK cells were treated with virus or virus and peptide mixtures (FP3). Plates were then washed, fixed and blocked, and any cell-associated virus detected with anti-influenza A antibody and HRP-conjugated secondary antibody. Increasing doses of peptide in the presence of 1.5% DMSO appears to inhibit adsorption of A/WSN/33 onto the cell surface of MDCK cells. Viral attachment was completely inhibited using 6 μg/ml of peptide, whereas no significant inhibition of attachments was observed when cells were treated with virus and vehicle (DMSO) alone. Based on this data we propose that the antiviral activity of the peptide is due to inhibition of viral attachment to cells. To determine whether peptide directly interacts with haemagglutinin (HA), 96 well microtiter plates were coated with increasing concentrations of peptide of the invention, washed, blocked with BSA, then incubated with 0.01 μg/well baculovirus-derived recombinant HA (California 04/2009 H1N1 Influenza A), which was detected with anti-Influenza A (strain USSR H1N1) antibody and an HRP-conjugated secondary antibody. The plates were developed using SigmaFAST OPD kit and read at 492 nm after addition of stop solution. Data are means and standard error of 3 replicates. FIG. 6 shows that FP1 and peptides derived therefrom bind to HA in a dose dependent manner with 50% inhibition occurring at 10 μg.

### EXAMPLE 6

### FP1 and Peptides Derived Therefrom Agglutinate Red Blood Cells

FP1 and its derivatives were determined to agglutinate red blood cells (RBCs) in a species-specific way. Both FP3 and FP4 were determined to agglutinate horse RBCs (end point 6.25 μg/ml) and human RBCs (end point 0.78 μg/ml). Both WSN and Udorn were determined to agglutinate human RBCs; however neither agglutinated horse RBCs. Human influenza viruses bind preferentially sialic acid containing N-acetylneuraminic acid alpha 2,6-galactose (SAalpha-2,6Gal) linkages while avian and equine viruses bind preferentially those containing N-acetylneuraminic acid alpha 2,3-galactose (SAalpha-2,3Gal) linkages. These data suggest that FP1 might compete with virus for sialic acid receptors.

### EXAMPLE 7

### Effect of WLVFFVIFYFFR on the Growth of Influenza Virus in a Mouse Model

Balb/c mice (female 5-6 weeks of age) were inoculated intranasally with 5×10³ pfu of WSN/33 (H1N1) and/or various concentrations of FP1 in 40 μl 2% DMSO in PBS. In the initial experiment a FP1 (WLVFFVIFYFFR) concentration of 1 μg was delivered at the same time as virus or on day 1 or day 3 post infection. The clinical signs of weight loss and condition/posture were measured daily and the virus yield in the lung was determined on day 7.

The results presented (FIG. 7) illustrate the potent antiviral activity of this peptide when given at the same time as virus. FP1 labelled with carboxyfluorosen has been seen in lung cells co-localising with virus at 7 days post infection and administration of the peptide (FIG. 9), indicating long-term persistence of the peptide in the lung environment.

In a second experiment (FIG. 8) FP2, FP4 and FP10 were compared for antiviral effects in vivo. Mice were administered 5×10³ pfu WSN and 20 μg of peptide intranasally and 7 days later lungs from 4 mice/group and viral titres determined by a plaque reduction assay. Whereas all three peptides showed efficient knock down of virus infectivity in vitro, only FP2 and FP4 were effective at inhibiting plaque formation in vivo. FP10 had values similar to the control groups.

### EXAMPLE 8

### Peptides of the Invention Protect Mice Against a Lethal Dose of Influenza Virus

To determine if the inhibition of influenza virus replication observed in vitro translated to protection in vivo, 5-6 week old BALB/c mice were inoculated intranasally with mouse adapted A/WSN/33 virus in the presence of peptide of the invention. Mice were monitored daily for clinical symptoms, weight was taken daily and virus titers obtained from day 7 lungs.

Pre-treatment of A/WSN/33 virus with 20 μg of FP4 peptide resulted in 100% survival of mice, with no clinical indications. Modified peptides maintained antiviral activity in vivo (FIG. 8) resulting in 2-4 log decreases in day 7 lung titers (determined by plaque reduction assays). Of note, neither FP2 nor FP4 peptides were immunogenic, determined by repeat administration of these peptides to BALB/c mice and determination of serum IgG levels by ELISA, even at 1:10 dilutions of serum. This model demonstrates potent anti-influenza virus activity against A/WSN/33 H1N1 virus when introduced prophylactically into the mouse respiratory tract. Therefore, peptides were able to protect mice from lethal H1N1 infection.

### EXAMPLE 9

### Assessment of Anti-Influenza Activity by Viral Growth Reduction Assays

To further investigate the antiviral activity of the peptides of the invention, their ability to inhibit the yield of infectious virus in culture supernatants was assessed. MDCK cells were treated with A/WSN/33 H1N1 virus and vehicle

(DMSO), or virus and peptide for 24 hours, and virus yield in supernatants determined by plaque assays. When infections were carried out in the continued presence of 1 μg/ml of FP4 peptide there was a 99% reduction in virus yield at 24 hours post-infection, when compared to infections in the presence of vehicle (DMSO) alone, similar to those obtained in peptide plaque reduction assays. No virus was detected 24 hours after treatment in presence of FP4 conjugated with polyethylene glycol (PEG). Of note, both FP4 and FP4-PEG had similar profiles when tested in standard peptide plaque reduction assays against A/WSN/33 virus. Infection in the presence of 1 μg/well of FP2 resulted in 97% reduction in virus yield, and an 85% reduction in presence of 1 μg/well FP1-D. Therefore, these peptides are highly effective at inhibiting the production of infectious A/WSN/33 H1N1 virus when administered simultaneously within an in vitro model system.

To assess the therapeutic potential of the peptides, MDCK cells were treated with A/WSN/33 H1N1 virus and vehicle (DMSO) followed 4 hours post-infection by the addition of either 1 μg/well FP4, FP4-PEG or FP1-D peptides. The virus yield in supernatants collected 24 hours post-infection were determined by plaque reduction assays. When FP4 was given simultaneously with virus it reduced virus yield at 24 hours by 99%, when peptide was added 4 hours post-infection the virus yield was reduced by 50%, suggesting that the FP4 was not able to inhibit subsequent viral infections as efficiently, possibly due to reduced availability of the peptide as a result of degradation or uptake by cells. However, treatment of virus-infected cells 4 hours post-infection with either pegylated FP4 or the FP1-D peptide resulted in a 68% reduction in the virus yield at 24 hours, as compared to the virus and vehicle alone control. Pegylation can enhance peptides and other potential pharmaceutical agents, protecting against degradation by proteolytic enzymes, increasing solubility as well as bioavailability. In the in vitro model, pegylation of the FP4 peptide or the use of D-amino acids resulted in enhanced antiviral activity when given therapeutically, probably due to an increase in availability and peptide half-life.

### EXAMPLE 10

### Agents of the Invention do not Appear to Interact with Sialic Acid

Although not wishing to be bound by initial results, it appears that peptides of the invention did not inhibit the attachment to cells through an interaction with sialic acid, as the presence of peptide did not inhibit the ability of influenza virus to agglutinate red blood cells. Indeed, peptides agglutinated RBCs, although the mechanism for this is unclear. Treatment of RBCs with bacterial sialidase to remove sialic acid receptors eliminated the ability to agglutinate these cells with virus, but not with peptide. Therefore, this suggests that sialic acid does not play a role in the virus/peptide interaction.

These results support the prophylactic use of peptides of the invention, for example into the respiratory tract, to reduce intranasal influenza A virus titres in the lung upon intranasal challenge. This leads to a reduction in the severity of infection, as indicated by weight gain in treated mice. The peptides also inhibit entry of parainfluenza viruses that use sialic acid as a receptor. Based on the experiments undertaken the peptides of the invention appear to be non-toxic, non-immunogenic and have not generated resistant virus variants.

Further, they support application of such peptides extend to animal populations susceptible to influenza virus, both to safeguard animal health and reduce the threat of zoonosis.

Although the invention has been particularly shown and described with reference to particular examples, it will be understood by those skilled in the art that various changes in the form and details may be made therein without departing from the scope of the present invention.

1. A method of treating a subject with a virus or preventing infection of said subject by a virus comprising administering to said subject an effective amount of an agent of formula A comprising a peptide having an amino acid sequence X1-X2-X3-X4-X5-X6, (SEQ ID NO 1) wherein:
X1 can be phenylalanine, isoleucine or tryptophan;
X2 can be leucine or phenylalanine or alanine;
X3 can be tyrosine or valine;
X4 can be leucine, phenylalanine or isoleucine;
X5 can be phenyalanine or alanine; and
X6 can be valine, arginine or tyrosine, or a fragment or variant of the peptide wherein said peptide fragment or variant is capable of specifically binding to haemagglutinin, to inhibit the binding of a virus having haemagglutinin on its surface to a cell.

2. The method as claimed in claim 1, wherein the agent of formula A comprises a peptide having at least 4 consecutive amino acids of WLVFFVIAYFAR (FP2) or WLVFFVIFYFFR (FP1) or a variant of the peptide wherein said peptide variant is capable of specifically binding to haemagglutinin.

3. The method as claimed in claim 1, wherein the agent of formula A comprises a peptide variant comprising between 1 and 3 amino acids conservatively substituted with another amino acid characterised in that the peptide can specifically bind to haemagglutinin.

4. The method as claimed in claim 1, wherein the agent of formula A comprises a peptide having at least 4 consecutive amino acids of WLVFFVIAYFAR (FP2) or a variant of the peptide wherein the peptide variant comprises between 1 and 3 amino acids conservatively substituted with another amino acid characterised in that the peptide can specifically bind to haemagglutinin.

5. The method as claimed in claim 1, wherein the agent comprises a peptide having an amino acid sequence FFVIFY (SEQ ID NO 2) or WLVFFV (SEQ ID NO 5), wherein optionally SEQ ID NO 2 or SEQ ID NO 5 can further include RRKK (SEQ ID NO 3) at the C or N terminal end of the peptide.

6. The method as claimed in claim 1, wherein the agent comprises a peptide having an amino acid sequence selected from:
SEQ ID NO 8
IFYFFR,
and
SEQ ID NO 10
IAYFAR.

7. The method as claimed in claim 1, wherein the agent comprises a peptide comprising an amino acid sequence selected from

| SEQ ID NO 11 | |
|---|---|
| FP2 | WLVFFVIAYFAR, |
| SEQ ID NO 12 | |
| FP3 | WLVFFVIFYFFRRRKK, |
| SEQ ID NO 13 | |
| FP4 | RRKKWLVFFVIYFFR, |
| SEQ ID NO 6 | |
| FP8 | WLVFFVRRKK, |
| SEQ ID NO 7 | |
| FP9 | FFVIFYRRKK, |
| SEQ ID NO 14 | |
| FP10 | IVWFYLFRFFVF, |
| SEQ ID NO 15 | |
| FP11 | FFVIAYRRKK, |
| SEQ ID NO 16 | |
| FP12 | FFVIAYFAR, |
| SEQ ID NO 17 | |
| FP13 | FFVIAYFARRRKK, |

or a fragment or variant of an amino acid sequence as provided by SEQ ID No 11, 12, 13, 6, 7, 14, 15, 16 or 17 capable of binding specifically to haemagglutinin and inhibiting the binding of virus to a cell.

8. The method as claimed in claim 7, wherein the agent comprises a peptide having an amino acid sequence selected from:

| SEQ ID NO 11 | |
|---|---|
| FP2 | WLVFFVIAYFAR, |
| SEQ ID NO 12 | |
| FP3 | WLVFFVIFYFFRRRKK, |
| SEQ ID NO 13 | |
| FP4 | RRKKWLVFFVIYFFR, |
| SEQ ID NO 14 | |
| FP10 | IVWFYLFRFFVF, |

or a fragment or variant of an amino acid sequence as provided by SEQ ID No 11 to 14 capable of binding specifically to haemagglutinin and inhibiting the binding of virus to a cell.

9. The method as claimed in claim 8, wherein the agent comprises a peptide having an amino acid sequence selected from
SEQ ID NO 12
FP3 WLVFFVIFYFFRRRKK,
SEQ ID NO 11
FP2 WLVFFVIAYFAR,
SEQ ID NO 13
FP4 RRKKWLVFFVIYFFR,
or a fragment or variant of an amino acid sequence as provided by SEQ ID No 11 to 13 capable of binding specifically to haemagglutinin and inhibiting the binding of virus to a cell.

10. The method as claimed in claim 9, wherein the agent comprises a peptide having an amino acid sequence selected from
SEQ ID NO 12
FP3 WLVFFVIFYFFRRRKK,
SEQ ID NO 13
FP4 RRKKWLVFFVIYFFR,
or a fragment or variant of an amino acid sequence as provided by SEQ ID No 12 or 13 capable of binding specifically to haemagglutinin and inhibiting the binding of virus to a cell.

11. The method as claimed in claim 9, wherein the agent comprises a peptide having an amino acid sequence WLVFFVIAYFAR (SEQ ID NO 11).

12. The method as claimed in claim 1, wherein the agent consists of a peptide having at least 4 consecutive amino acids of WLVFFVIAYFAR (FP2) or WLVFFVIFYFFR (FP1) or a variant of the peptide wherein said peptide variant is capable of specifically binding to haemagglutinin for use in the treatment of virus having haemagglutinin on its surface.

13. The method as claimed in claim 12, wherein the agent consists of a peptide having an amino acid sequence selected from:

| SEQ ID NO 11 | |
|---|---|
| FP2 | WLVFFVIAYFAR, |
| SEQ ID NO 12 | |
| FP3 | WLVFFVIFYFFRRRKK, |
| SEQ ID NO 13 | |
| FP4 | RRKKWLVFFVIYFFR, |
| SEQ ID NO 6 | |
| FP8 | WLVFFVRRKK, |
| SEQ ID NO 7 | |
| FP9 | FFVIFYRRKK, |
| SEQ ID NO 14 | |
| FP10 | IVWFYLFRFFVF, |
| SEQ ID NO 15 | |
| FP11 | FFVIAYRRKK, |
| SEQ ID NO 16 | |
| FP12 | FFVIAYFAR, |
| and | |
| SEQ ID NO 17 | |
| FP13 | FFVIAYFARRRKK. |

14. The method as claimed in claim 1 for use in the treatment of virus having haemagglutinin on its surface to a cell, wherein said virus is influenza.

15-26. (canceled)

27. An agent of formula A comprising or consisting of a peptide having an amino acid sequence selected from any one of SEQ ID NOs 1 to 17, 21, 22, or 23.

28. The agent as claimed in claim 27 wherein the agent of formula A comprises or consists of a peptide having an amino acid sequence having at least 4 consecutive amino acids of WLVFFVIAYFAR (FP2) or a variant of the peptide wherein the peptide variant comprises between 1 and 3 amino acids conservatively substituted with another amino acid characterised in that the peptide can specifically bind to haemagglutinin.

29. The agent of formula A as claimed in claim 27 comprising or consisting of a peptide having an amino acid sequence selected from any one of SEQ ID NOs 2 to 17, 21, 22, or 23.

30. A nucleic acid sequence which can encode a peptide as claimed in claim 27.

31. An expression construct comprising a nucleic acid sequence of claim 30 and a promoter region operably linked to the nucleic acid sequence.

32. A method to determine an agent which includes a peptide of formula A or a peptide or non-peptide based on formula A, wherein formula A comprises an amino acid sequence X1-X2-X3-X4-X5-X6, (SEQ ID NO 1) wherein X1 can be phenylalanine, isoleucine or tryptophan;
X2 can be leucine or phenylalanine or alanine;
X3 can be tyrosine or valine;
X4 can be leucine, phenylalanine or isoleucine;
X5 can be phenyalanine or alanine; and
X6 can be valine, arginine or tyrosine,
comprising the steps
a) exposing test cells to virus in the presence and absence of the agent to be tested under conditions which would typically allow virus to infect such test cells,
b) comparing the number of infected test cells and/or rate of infection of the test cells following exposure to the virus, and
c) determining whether the agent to be tested provides a protective effect and inhibits infection of a cell.

33-35. (canceled)

36. A cell comprising a nucleic acid able to encode an agent comprising a peptide as claimed in claim 27.

37. The cell of claim 36 wherein the cell is a cell found in indigenous microflora of a subject.

38. The cell as claimed in claim 36 wherein the cell is Lactobacillusin particular Lactobacillus provided in a probiotic culture suitable for administration to a subject.

39. An animal comprising a cell as claimed in claim 36 wherein an agent as claimed in claim 27 is expressed in the animal.

40. The animal as claimed in claim 39 wherein the animal has been genetically modified such that an agent as claimed in claim 27 is expressed in the animal.

41. The animal as claimed in claim 39 wherein the animal is selected from poultry, a domesticated animal, in particular a chicken or pig.

42. A pharmaceutical composition comprising an agent as claimed in claim 27, a nucleic acid of claim 30, an expression construct of claim 31 or a cell of claim 36 in combination with a pharmaceutically acceptable carrier.

43-44. (canceled)
